(19) United States
(12) Patent Application Publication
(10) Pub. No.: US 2006/0265765 A1
(43) Pub. Date: Nov. 23, 2006

(54) Oncogene HOXB13 and its siRNAs

(75) Inventors: Toshinori Agatsuma, Saitama-shi (JP); Keisuke Fukuchi, Tokyo (JP); Satoko Nishimura, Urayasu-shi (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: SANKYO COMPANY, LIMITED, Tokyo (JP)

(21) Appl. No.: 11/134,165

(22) Filed: May 19, 2005

(51) Int. Cl. A01K 67/027; C12N 5/06; A61K 48/00

(52) U.S. Cl. 800/10; 514/44; 800/18; 435/354

(57) ABSTRACT

A method of producing cancer cells using an oncogene, a method of detecting cancer using the cancer cell, a method of screening compounds having cancer therapeutic and/or preventive effects and a cancer therapeutic and/or preventive pharmaceutical composition. There could be provided a method of detecting cancer using the expression of HOXB13 as an indicator wherein HOXB13 gene is excessively expressed in a cell, and a cell line in which cell growth ability is changed is selected; a method of screening compounds having novel cancer therapeutic and/or preventive effects by using OXB13; a pharmaceutical composition; and a method of treating cancer using said pharmaceutical composition.

### BACKGROUND OF THE INVENTION

1. Field of the Invention

The present invention relates to a cancer therapeutic and/or preventive pharmaceutical composition and a method of producing cancer cells using an oncogene.

2. Background Art

Cancer continues to be one of the leading causes of death in the US, Europe, Japan and other advanced countries. Clinically, although various medical procedures are employed, including surgical removal of cancer tissue, radiotherapy and chemotherapy, these cannot be said to be based on an adequately accurate understanding of malignant transformation and growth of cancer cells, and in many cases, their effects are only partial, and cannot satisfy the majority of patients. With respect to the malignant transformation and the mechanisms of growth of cancer cells, extensive research has been conducted at numerous research institutions for ten and several years.

At present, the growth of normal cells is thought to be controlled according to the balance between oncogenes (growth promoting genes) and tumor suppressor genes (Nature Medicine, Vol. 10, 2004, p. 789-799).

Although cellular endogenous oncogenes themselves typically do not always have potent effects that cause canceration of normal cells, if their regulatory function is lost due to structural mutation, they become direct factors of abnormal growth and canceration.

Examples of these oncogenes include Ras, which causes cancer as a result of a point mutation, and Abl gene, which causes abnormal acceleration of enzyme activity due to a chromosomal aberration.

In addition, there are also many cases in which canceration is triggered by accelerated expression of an oncogene without the occurrence of a mutation. For example, accelerated expression due to gene amplification of the growth factor receptor HER2/neu is induced in metastatic breast cancer, and its inhibitor is used for treatment of metastatic breast cancer.

The aforementioned facts strongly suggest the working hypothesis that these oncogenes provide extremely valid target molecules for use as targets of cancer therapy.

Among cancer diseases, prostate cancer in particular is the most frequently diagnosed cancer in the US, and is the second leading cause of cancer death among men.

Roughly 300,000 men are initially diagnosed with prostate cancer each year, and more than 40,000 men die as a result of this disease.

Even though death due to prostate cancer is mainly caused by a metastasized disease, nearly 60% of patients first diagnosed with this cancer have primary tumors localized in the prostate gland.

Although surgery and radiotherapy are frequently effective for patients with such localized cancers, nearly all patients in which the tumor has disseminated and metastasized are untreatable.

Although extensive research has been conducted thus far, there is very little known regarding the biological mechanism that causes the onset and progression of prostate cancer.

As has been described above, findings regarding oncogenes involved in the onset and progression of these cancers (including prostate cancer) are extremely useful in terms of elucidating their mechanisms and developing therapeutic drugs.

Alternative methods for determining the effects resulting from functional inhibition of target molecules involve the use of techniques that lower the expression of their gene by some method.

Examples of such methods include the use of antisense oligonucleotides, which are single-stranded DNAs having a sequence complementary to a target gene, and ribozymes, which are single-stranded RNAs having RNA splicing activity.

In addition to these methods, RNA interference (RNAi), which uses double-stranded RNA (dsRNA), has recently come to be used (Microbiology and Molecular Biology Reviews, 2003, Vol. 67, p. 657-685).

RNA interference (RNAi) is a phenomenon in which homologous mRNA present within cells is specifically degraded by dsRNA, and was reported in 1998 as a phenomenon that occurs in nematodes (Nature, 1998, Vol. 391, p. 806-811).

In mammals, since long-strand dsRNA ends up causing an interferon response (Microbiology and Molecular Biology Reviews, 1998, Vol. 62, p. 1415-1434, The International Journal of Biochemistry and Cell Biology, 1997, Vol.29, p. 945-949), it was initially thought that it would be difficult to utilize the effects of RNAi as a specific gene suppression technique. However, it was reported in 2001 by Elbashir et al. that RNAi effects can be induced while avoiding an interferon response by utilizing an intermediate product of RNAi in the form of short dsRNA of 21 to 23 mer (Genes and Developments, 2001, Vol. 15, p.188-200). RNAi resulting from this siRNA is attracting attention as a simple and potent gene expression suppression technique unlike anything thus far. In addition, siRNA that targets growth factors of cancer cells has been reported to yield antitumor effects by administering to mouse tumors after mixing with atherocollagen and cationic liposomes (Nucleic Acids Research, 2004, Vol. 32, e. 109: online publication), and numerous attempts have been made to utilize siRNA as a therapeutic drug.

Homeobox (HOX) is a gene cluster that was discovered in experiments on fruit flies that is an important transcription gene cluster for organ formation during embryogenesis.

This gene cluster consists of genes arranged in a row that regulate the expression of function of other genes by controlling the transcription of those genes by acting in the order in which they are arranged.

Mutations of this gene cluster are known to cause the appearance of large-scale morphological changes such as the thoracic region changing to the abdominal region, disappearing or overlapping of body segments and the lower lip becoming an antenna or limb.

Homeobox gene clusters having a high degree of homology with that found in flies have been discovered in an extremely large number of animals ranging from planaria to sea urchins, nematodes and humans, and these gene clusters are believed to control organogenesis by regulating the expression of various genes even in higher animals including humans.

Within the Homeobox gene cluster, the HOXB13 gene was identified in 1996 from the cDNA library of human cervical adenocarcinoma cell line HeLa as a new HOX gene similar to the Abdominal B subgroup said to be involved in the formation of urogenitalia (Development, 1996, Vol. 122, p. 2475-2484).

Expression of HOXB13 has been observed in the spinal cord, hindgut and urogenital sinus of mouse fetuses.

In mature individuals, expression has been observed in the prostate gland and large intestine, and expression in the prostate gland has been reported to be non-androgen-dependent (Prostate, 1999, Vol. 41, p. 203-207).

Reports have been published in the past indicating the involvement of several HOX genes with canceration (Cell Growth Differentiation, 1993, Vol. 4, p. 431-441; Molecular and Cellular Biology, 1991, Vol. 11, p. 554-557; Prostate, 1996, Vol. 29, p. 395-398; Cancer Research, 1994, Vol. 54, p. 5981-5985; Journal of Cancer, 1992, Vol. 51, p. 892-897; International Journal of Hematology, 1998, Vol. 68, p. 343-353). HOXB13 has been reported to be a gene marker expressed in prostate cancer (British Journal of Cancer, 2004, Vol. 7, online publication). In addition, expression of HOXB13 gene has been reported to be high at the affected sites in breast cancer patients unresponsive to tamoxifen therapy, while cell mobility and invasiveness have been reported to be increased in MCF10A normal mammary gland epithelial cells inserted with HOXB13 gene (Cancer Cell, 2004, Vol. 5, p. 607-616). However, there have been no reports that have actually suggested a direct correlation with the growth of cancer cells by suppressing or inhibiting HOXB13 gene or its gene products.

### SUMMARY OF THE INVENTION

The object of the present invention is to discover a gene relating to cancer cells and the onset and/or growth of cancer cells, develop a method of producing cancer cells using said oncogene, and provide a cancer therapeutic and/or preventive pharmaceutical composition, and a method of treating cancer using said pharmaceutical composition.

The inventors of the present invention found that cancer cells can be produced by excessively expressing the HOXB13 gene, and provided a substance that suppresses the expression of HOXB13, thereby leading to completion of the present invention.

Namely, the present invention is comprised of:

(1) a cancer therapeutic and/or preventive pharmaceutical composition comprising an oligonucleotide having at least one of the nucleotide sequences selected from the group consisting of the following 1) to 4), or a nucleotide sequence complementary to a partial sequence of said sequence:
1) the nucleotide sequence containing nucleotide numbers 177 to 1031 of SEQ ID NO: 1 of the Sequence Listing,
2) the nucleotide sequence containing nucleotide numbers 131 to 985 of SEQ ID NO: 3 of the Sequence Listing,
3) the nucleotide sequence containing nucleotide numbers 55 to 909 of SEQ ID NO: 5 of the Sequence Listing,
4) the nucleotide sequence containing nucleotide numbers 87 to 941 of SEQ ID NO: 7 of the Sequence Listing,

(2) a cancer therapeutic and/or preventive pharmaceutical composition comprising an antibody which specifically recognizes HOXB13,

(3) a cancer therapeutic and/or preventive pharmaceutical composition comprising a siRNA for at least one nucleotide sequence selected from the group consisting of the following 1) to 4) or a partial sequence of said sequence:
1) the nucleotide sequence containing nucleotide numbers 177 to 1031 of SEQ ID NO: 1 of the Sequence Listing,
2) the nucleotide sequence containing nucleotide numbers 131 to 985 of SEQ ID NO: 3 of the Sequence Listing,
3) the nucleotide sequence containing nucleotide numbers 55 to 909 of SEQ ID NO: 5 of the Sequence Listing,
4) the nucleotide sequence containing nucleotide numbers 87 to 941 of SEQ ID NO: 7 of the Sequence Listing;

(4) a siRNA selected from the group consisting of the following 1) to 5):
1) a siRNA comprising the combination of an oligonucleotide composed of the nucleotide sequence of SEQ ID NO: 13 of the Sequence Listing, and an oligonucleotide composed of the nucleotide sequence of SEQ ID NO: 14 of the Sequence Listing;
2) a siRNA comprising the combination of an oligonucleotide composed of the nucleotide sequence of SEQ ID NO: 15 of the Sequence Listing, and an oligonucleotide composed of the nucleotide sequence of SEQ ID NO: 16 of the Sequence Listing;
3) a siRNA comprising the combination of an oligonucleotide composed of the nucleotide sequence of SEQ ID NO: 17 of the Sequence Listing, and an oligonucleotide composed of the nucleotide sequence of SEQ ID NO: 18 of the Sequence Listing;
4) a siRNA comprising the combination of an oligonucleotide composed of the nucleotide sequence of SEQ ID NO: 27 of the Sequence Listing, and an oligonucleotide composed of the nucleotide sequence of SEQ ID NO: 28 of the Sequence Listing;
5) a siRNA comprising the combination of an oligonucleotide composed of the nucleotide sequence of SEQ ID NO: 29 of the Sequence Listing, and an oligonucleotide composed of the nucleotide sequence of SEQ ID NO: 30 of the Sequence Listing;

(5) a cancer therapeutic and/or preventive pharmaceutical composition comprising at least one siRNA selected from the group consisting of the following 1) to 5):
1) a siRNA comprising the combination of an oligonucleotide composed of the nucleotide sequence of SEQ ID NO: 13 of the Sequence Listing, and an oligonucleotide composed of the nucleotide sequence of SEQ ID NO: 14 of the Sequence Listing;
2) a siRNA comprising the combination of an oligonucleotide composed of the nucleotide sequence of SEQ ID NO: 15 of the Sequence Listing, and an oligonucleotide composed of the nucleotide sequence of SEQ ID NO: 16 of the Sequence Listing;
3) a siRNA comprising the combination of an oligonucleotide composed of the nucleotide sequence of SEQ ID NO: 17 of the Sequence Listing, and an oligonucleotide composed of the nucleotide sequence of SEQ ID NO: 18 of the Sequence Listing;
4) a siRNA comprising the combination of an oligonucleotide composed of the nucleotide sequence of SEQ ID NO: 27 of the Sequence Listing, and an oligonucleotide composed of the nucleotide sequence of SEQ ID NO: 28 of the Sequence Listing;
5) a siRNA comprising the combination of an oligonucleotide composed of the nucleotide sequence of SEQ ID NO: 29 of the Sequence Listing, and an oligonucleotide composed of the nucleotide sequence of SEQ ID NO: 30 of the Sequence Listing;

(6) a pharmaceutical composition according to any one of (1) to (3) and (5), wherein the cancer is prostate cancer,

(7) a method of treating cancer comprising administering to a human in need thereof a pharmaceutically effective amount of the pharmaceutical composition according to any one of (1) to (3) and (5);

(8) the method of treating cancer according to (7), wherein the cancer is prostate cancer;

(9) a method of producing cancer cells comprising the following steps 1) and 2):
1) a step of transforming cells using a polynucleotide described in any one of (1) to (5) below:
   (1) a polynucleotide composed of the nucleotide sequence containing nucleotides 177 to 1031 of SEQ ID NO: 1 of the Sequence Listing,
   (2) a polynucleotide composed of the nucleotide sequence containing nucleotides 131 to 985 of SEQ ID NO: 3 of the Sequence Listing,
   (3) a polynucleotide composed of the nucleotide sequence containing nucleotides 55 to 909 of SEQ ID NO: 5 of the Sequence Listing,
   (4) a polynucleotide composed of the nucleotide sequence containing nucleotides 87 to 941 of SEQ ID NO: 7 of the Sequence Listing,
   (5) a polynucleotide that hybridizes under stringent conditions with a polynucleotide composed of a nucleotide sequence complementary to a polynucleotide described in any one of (1) to (4) above, and is composed of a nucleotide sequence that encodes a protein substantially identical to HOXB13, and
2) a step of selecting a cell line in which a change in cell growth ability has occurred as a result of step 1);

(10) the method of producing cancer cells according to (9), wherein the cells are animal cells;

(11) the method of producing cancer cells according to (10), wherein the animal cells are derived from mammal;

(12) the method of producing cancer cells according to (11), wherein the mammal is a human, monkey, mouse or rat;

(13) the method of producing cancer cells according to (11), wherein the mammal is a human;

(14) the method of producing cancer cells according to (9), wherein the cells are mouse fibroblast cell line NIH3T3 cells;

(15) the method of producing cancer cells according to any one of (9) to (14), wherein cells are transformed with a recombinant vector;

(16) cancer cells obtained by the method of producing cancer cells according to any one of (9) to (15);

(17) a non-human mammal introduced with the cancer cells according to (16).

### BRIEF DESCRIPTION OF THE DRAWINGS

FIG. 1 shows the formation of colonies by a HOXB13 expression strain. In the panel identified as “parent NIH3T3 cell” only 2.0±2.0 (average ± standard deviation) colonies were observed in the parent NIH3T3 cell. The panel identified as “HOXB13 expressing cell” shows that 1999.3±239.2 (average ± standard deviation) colonies were detected in the HOXB13 expressing cell line NIH3T3. These results show that HOXB13 has a colony inducing activity.

FIG. 2 shows a spheroid growth test.

FIGS. 3-1 to 3-7 show the gene expression suppression effect of siRNA-HOXB13 on HOXB13 stably expressing strain.

FIG. 3-1 comprises graphs showing the gene expression suppression effect of siRNA-HOXB13 on a HOXB13 stably expressing strain.

FIG. 3-2 is a graph showing the gene expression suppression effect of the introduction of siRNA-Eng5 on a HOXB13 stably expressing strain.

FIG. 3-3 is a graph showing the gene expression suppression effect of the introduction of siRNA-HOXB13 No. 1 (“siRNA No. 1”)on a HOXB13 stably expressing strain.

FIG. 3-4 is a graph showing the gene expression suppression effect of the introduction of siRNA-HOXB13 No. 2 (“siRNA No. 2”) on a HOXB13 stably expressing strain.

FIG. 3-5 is a graph showing the gene expression suppression effect of the introduction of siRNA-HOXB13 No. 3 (“siRNA No. 3”) on a HOXB13 stably expressing strain.

FIG. 3-6 is a graph showing the gene expression suppression effect of the introduction of siRNA-HOXB13 No. 4 (“siRNA No. 4”) on a HOXB13 stably expressing strain.

FIG. 3-7 is a graph showing the gene expression suppression effect of the introduction of siRNA-HOXB13 No. 5 (“siRNA No. 5”) on a HOXB13 stably expressing strain.

FIG. 4 is a graph showing the growth suppression effect of HOXB13-siRNA on hormone non-responsive prostate cancer cell line PC-3.

FIG. 5 shows colony formation of the HOXB13 gene expressing NIH3T3 cell in soft agar. The panel labeled as “NIH3T3-mock” shows that a small number of MTT stained cells were observed in the NIH3T3 mock. The panel labeled as “NIH3T3-HOXB13” shows that more MTT stained cells were observed in the NIH3T3-HOXB13 strain than in NIH3T3-mock.

### DETAILED DESCRIPTION OF THE INVENTION

As a result of discovering HOXB13, which is specifically expressed in prostate cancer, and that said gene is involved in the onset and/or growth of cancer cells, the present invention is able to provide a cancer therapeutic and/or preventive pharmaceutical composition, and a method of treating cancer using said pharmaceutical composition.

In the present specification, a compound having cancer therapeutic and/or preventive effects refers to a compound having activity that suppresses cancer growth, activity that reduces cancer, and/or activity that prevents the onset of cancer. In the present specification, “cancer” and “tumor” are used with the same meaning. In the present specification, the term “gene” includes not only DNA, but also mRNA, cDNA and its cRNA. Thus, HOXB13 DNA, mRNA, cDNA and cRNA are included in the “HOXB13 gene” in the present invention. In the present specification, the term “polynucleotide” has the same meaning as nucleic acid, and includes DNA, RNA, probes, oligonucleotides and primers. In the present specification, “polypeptide” and “protein” are used without distinction. In addition, in the present specification, “RNA fraction” refers to a fraction that contains RNA. Moreover, in the present specification, cells of individual animals and cultured cells are included in the term “cells”. In the present specification, “cell canceration” refers to a cell demonstrating abnormal growth such as losing sensitivity to contact inhibition of cell growth or demonstrating anchorage-independent growth, and cells that demonstrate this abnormal growth are referred to as “cancer cells”. In the present specification, a “substantially identical protein” refers to a protein having an identical function such as cell canceration activity possessed by HOXB13. Furthermore, the term “oncogene” in the present invention includes oncogenes as well as proto-oncogenes.

1. Acquisition of the HOXB13 Gene

The nucleotide sequence of cDNA of the human HOXB13 used in the present invention is indicated with, for example, nucleotide numbers 1 to 1316 of SEQ ID NO: 1 of the Sequence Listing, nucleotide numbers 1 to 1270 of SEQ ID NO: 3, nucleotide numbers 1 to 1026 of SEQ ID NO: 5, or nucleotide numbers 1 to 1356 of SEQ ID NO: 7. In addition, human HOXB13 is a protein encoded by, for example, nucleotide numbers 177 to 1031 of SEQ ID NO: 1 of the Sequence Listing, nucleotide numbers 131 to 985 of SEQ ID NO: 3, nucleotide numbers 55 to 909 of SEQ ID NO: 5, or nucleotide numbers 87 to 941 of SEQ ID NO: 7.

The amino acid sequence of human HOXB13 is indicated with, for example, amino acid numbers 1 to 284 of SEQ ID NO: 2 of the Sequence Listing, amino acid numbers 1 to 284 of SEQ ID NO: 4, amino acid numbers 1 to 284 of SEQ ID NO: 6, or amino acid numbers 1 to 284 of SEQ ID NO: 8. In addition, the nucleotide sequence of human HOXB13 cDNA is registered with GenBank under accession number U81599 (version: U81599.1), accession number NM_{—}006361 (version: NM_{—}006361.2), accession number U57052 (version: U57052.1) and accession number BC007092 (version: BC007092.1). In the present specification, “HOXB13 gene” refers to a gene comprising a nucleotide sequence containing any one of nucleotide numbers 177 to 1031 of SEQ ID NO: 1 of the Sequence Listing, nucleotide numbers 131 to 985 of SEQ ID NO: 3, nucleotide numbers 55 to 909 of SEQ ID NO: 5, or nucleotide numbers 87 to 941 of SEQ ID NO: 7, or a gene comprising a nucleotide sequence that hybridizes under stringent conditions with a polynucleotide composed of a nucleotide sequence complementary to a gene composed of said nucleotide sequence, and encodes a protein having biological activity identical to HOXB13. In addition, in the present specification, “HOXB13” refers to a protein in which the amino acid sequence comprises any one of the amino acid sequences indicated with amino acid numbers 1 to 284 of SEQ ID NO: 2 of the Sequence Listing, amino acid numbers 1 to 284 of SEQ ID NO: 4, amino acid numbers 1 to 284 of SEQ ID NO: 6, or amino acid numbers 1 to 284 of SEQ ID NO: 8, or a protein comprising an amino acid sequence in which one or several amino acids in said protein amino acid sequence have been deleted, substituted or added, and has biological activity identical to HOXB13. The term “total RNA fraction” in the present specification refers to a fraction that contains total RNA, and is a fraction that contains total RNA that has been extracted using ordinary methods such as a solvent for extraction of RNA from blood, various organs, various tissues or cultured cells and so forth.

The HOXB13 gene can be acquired by the methods indicated below.

(1) Case of Using Human cDNA Library

Full-length cDNA is acquired from a cDNA library that expresses the HOXB13 gene in accordance with a known method such as colony hybridization. Human HOXB13 cDNA is able to be acquired by PCR using this full-length cDNA as a template. A cDNA library derived from human prostate cancer, human lung cancer, human breast cancer, human stomach cancer, human colon cancer, human malignant melanoma, human pancreatic cancer and their respective non-cancerated normal tissues can be used for the cDNA library. As a commercially available human DNA library, prostate-Leiomyosarcoma cDNA (Invitrogen: 11598-018), Fetal Brain cDNA (Invitrogen: 10662-013), Marathon-Ready cDNA, Normal Prostate, pooled (CLONTECH Laboratories, Inc.: 7418-1-1) can be used, or it can be prepared by themselves. Furthermore, HOXB13 CDNA can also be acquired by carrying out PCR directly using a cDNA library as a template. The PCR primer should be able to amplify HOXB13 cDNA, and suitable primers can be selected using known methods. Polynucleotides having, for example, the following nucleotide sequences can be selected as PCR primers that amplify HOXB13 cDNA.

5′-caccatggagcccggcaattatgcca-3′
(Primer 1: SEQ ID NO: 9 of the Sequence Listing)
and,
5′-ttaaggggtagcgctgttctt-3′
(Primer 2: SEQ ID NO: 10 of the Sequence Listing).

The following describes an example of a method of preparing cDNA.

When extracting the total RNA fraction from blood, various tissues or various organs removed from humans, the blood, tissue or organ is preferably dissolved directly with a solvent for RNA extraction (for example, that containing a component having an action that deactivates ribonuclease such as phenol). Alternatively, cells are recovered by a method such as carefully scraping with a scraper so as not to damage the tissue cells, or gently extracting the cells from tissue using a protease such as trypsin, followed promptly by an RNA fraction extraction step.

Although examples of methods employed to extract an RNA fraction include the guanidine thiocyanate-cesium chloride ultracentrifugation, guanidine thiocyanate-hot phenol, guanidine hydrochloride and acidic guanidine thiocyanate-phenol-chloroform methods (Chomczynski, P. and Sacchi, N., (1987) Anal. Biochem., 162, 156-159), the acidic guanidine thiocyanate-phenol-chloroform method is used preferably. In addition, commercially available RNA extraction reagents (such as ISOGEN (Nippon Gene) or TRIzol Reagent (Invitrogen) can also be used in accordance with the protocol provided with the reagent. For example, the total RNA fraction can be extracted from human prostate cancer cell line LNCaP (ATCC (American Tissue Culture Collection) No. CRL-1740) using TRIzol reagent.

The resulting total RNA fraction is preferably used after additionally purifying to mRNA only as necessary. Although there are no limitations on the purification method, since the majority of mRNAs present in the cytoplasm of eukaryotic cells are known to have a poly(A) sequence on their 3′ terminal, mRNA can be purified by utilizing this characteristic by, for example, adsorbing mRNA onto a biotinated oligo(dT) probe, capturing mRNA on paramagnetic particles on which streptoavidin has been immobilized and washing, followed by eluting the mRNA. In addition, a method can also be employed for purifying mRNA by adsorbing mRNA onto an oligo(dT) cellulose column followed by eluting the adsorbed mRNA. Moreover, mRNA can be further fractionated using, for example, sucrose density gradient centrifugation.

cDNA can be synthesized by a known method with reverse transcriptase using mRNA as a template. For example, cDNA can be synthesized using Omniscript Reverse Transcriptase (QIAGEN) in accordance with the protocol provided. HOXB13 cDNA can then be acquired by carrying out PCR on the resulting cDNA using PCR primers specific for amplification of the HOXB13 gene (for example, the combination of primers of SEQ ID NO: 9 and SEQ ID NO: 10 of the Sequence Listing). PCR can be carried out under ordinary reaction conditions.

Furthermore, a person with ordinary skill in the technical art to which the present invention belongs can prepare a polynucleotide having biological activity that promotes cell canceration equivalent to naturally-occurring HOXB13 gene by altering a portion of the naturally-occurring nucleotide sequence of human HOXB13 gene by altering such as substituting with another nucleotide or deleting or adding a nucleotide. In this manner, a polynucleotide having a nucleotide sequence in which a nucleotide in the naturally-occurring nucleotide sequence has been substituted, deleted or added, and which demonstrates variations in expression equivalent to naturally-occurring HOXB13 gene, can also be used in the present invention. The nucleotide sequence can be altered by methods such as introduction of a deletion using a restriction enzyme or DNA exonuclease, introduction of a mutation by site-specific mutagenesis, alteration of a nucleotide sequence by PCR using mutant primers or direct introduction of synthetic mutant DNA. In addition, a polynucleotide can also be used that hybridizes under stringent conditions with a polynucleotide comprising a nucleotide sequence complementary to the HOBX13 gene, and which has oncogene growth activity similar to HOXB13.

In the present invention, “hybridizes under stringent conditions” refers to hybridizing under conditions, or conditions equivalent thereto, which enable identification by hybridizing at 68° C. in a commercially available hybridization solution such as ExpressHyb Hybridization Solution (Clontech) or hybridizing at 68° C. in the presence of 0.7 to 1.0 M NaCl using a filter on which DNA has been immobilized, followed by washing at 68° C. using a 0.1× to 2× concentration SSC solution (a 1× concentration SSC solution is composed of 150 mM NaCl and 15 mM sodium citrate).

2. Expression of the HOXB13 Gene

An example of a method of expressing the HOXB13 gene in an animal individual consists of incorporating the resulting full-length cDNA in a virus vector followed by administration to the animal. Examples of gene introducing methods that use a virus vector include introduction following incorporation of the cDNA in a DNA virus such as retrovirus, adenovirus, adeno-associated virus, herpes virus, vaccinia virus, pox virus and polio virus, or an RNA virus incorporated with cDNA. In particular, methods using retrovirus, adenovirus, adeno-associated virus or vaccinia virus are preferable.

Examples of non-viral gene introduction methods include a method in which an expression plasmid is administered intramuscularly directly (DNA vaccination method), liposome method, lipofection method, microinjection method, calcium phosphate method and electroporation method, with the DNA vaccination method and liposome method being particularly preferable.

In addition, studies can also be made of the effects in cultured cells that appear on functions possessed by various target cells, and particularly cell canceration for example, cell morphology or cell differentiation, by introducing full-length cDNA into cells such as muscle cells, liver cells, fat cells or prostate cells derived from humans, mice, rats and so forth, or cells that differentiate into muscle cells, liver cells or fat cells (for example, fibroblasts), and highly expressing the full-length cDNA in those cells. Conversely, whether or not effects appear on the functions, morphology or differentiation of various target cells can be investigated by introducing an antisense nucleic acid with respect to the test gene into those cells.

In the introduction of full-length cDNA into animals or cells, said cDNA is incorporated into a vector that contains a suitable promoter and sequence involved in phenotypic expression, and host cells are transformed with said vector. Promoters located upstream from a gene to be normally expressed and promoters having an RNA splicing site, polyadenylation site or transcription termination sequence can be used as expression promoters of vertebrate cells, and these may also have a replication origin as necessary. Examples of said expression vectors include, but are not limited to, pSV2dhfr having an early promoter of SV40 (Subramani, S. et al. (1981) Mol. Cell. Biol. 1, p. 854-864), and retrovirus vectors pLNCX, pLNSX, pLXIN and pSIR (Clontech) . Said expression vectors can be incorporated into COS cells or mouse fibroblast line NIH3T3 (ATCC No. CRL-1658) and so forth by the diethylaminoethyl (DEAE)-dextran method (Luthman, H. and Magnusson, G. (1983) Nucleic Acids Res. 11, p. 1295-1308), calcium phosphate-DNA co-precipitation method (Graham, F. I. and van der Eb, A. J. (1973) Virology, 52, 456-457), electroporation (Neumann, E. et al. (1982) EMBO J., 1, p. 841-845), or Lipofectamine 2000 (Invitrogen), Lipofectamine PLUS (Invitrogen), DMRIE-C Reagent (Invitrogen), FuGENE6 (Roche Diagnostics) and so forth. Alternatively, in the case of a retrovirus vector, the virus can be produced by either incorporating in a packaging cell line such as 293-10A1 (IMGENEX) or PT67 (Clontech), or incorporating together with 293 cells (Takara Shuzo) or NIH3T3 cells in a packaging plasmid such as pCL-10A1 or pCL-Eco (IMGENEX), followed by infecting COS cells or mouse fibroblasts NIH3T3 to obtain the desired transformed cells.

In addition, a knockout animal can be produced in which a target gene has been destroyed in a normal animal by gene manipulation to study the effects that appear, such as on tumorigenesis and/or the tumor growth mechanism. Conversely, a transgenic animal can be produced so that a target gene is highly expressed in animal having a tumor to investigate the function of HOXB13 by observing morphological changes in the tumor. The transgenic animal can be obtained by sampling fertilized eggs from an animal and inserting the gene, followed by transplanting to a pseudo-pregnant animal and allowing to develop, and this procedure should be in accordance with a known method (see Developmental Engineering Laboratory Manual (Tatsuji Nomura, editorial supervisor, Motoya Katsugi, editor, published in 1987), and Japanese Patent Application (Kokai) No. Hei 5-48093). More specifically, in the case of mice, for example, after first administering an ovulation inducer to a female mouse, the female is mated with a male of the same strain, and a pronuclear fertilized egg is sampled from the uterine tube of the female mouse on the following day. Next, a solution of the DNA fragment to be inserted is injected into the prenucleus of the fertilized egg using a micropipette. Furthermore, there are no particular limitations on promoters, enhancers and other regulatory genes for expressing the gene to be inserted in animal cells provided they function in the cells of the animal in which the DNA fragment is to be introduced. The fertilized egg into which DNA has been injected is transplanted to the uterine tube of a pseudo-pregnant surrogate parent female mouse (such as Slc:ICR) after which the offspring is delivered by natural birth or Cesarean section about 20 days later.

Examples of methods of confirming that the resulting animal retains the introduced gene include a method in which DNA is extracted from the tail and so forth of the animal, followed by amplifying said DNA by PCR using sense and antisense primers specific to that DNA, and a method in which, after digesting the DNA with restriction enzyme, the resulting digested DNA is subjected to gel electrophoresis, and after blotting the DNA in the gel onto a Nylon membrane and so forth, southern blot analysis is carried out using all or a portion of the labeled introduced gene as a probe.

3. Production Method of Cancer Cells Using the HOXB13 Gene

Whether or not the target gene functions as an oncogene, namely whether or not the target gene has malignant transformation ability can be investigated according to the presence or absence of sensitivity to contact inhibition or anchorage independent growth in the cells in which it is expressed (Atsushi Yokota, Tadashi Yamamoto, ed., Bio Manual UP Series, Cancer Research Protocol, Yodosha Co., Ltd., p. 168-174 (Oct. 15, 1995). Namely, a gene that causes cells to lose sensitivity to contact inhibition or causes cells to exhibit anchorage independent growth can be said to be an oncogene.

For example, when the HOXB13 gene is excessively expressed in mouse fibroblast cell line NIH3T3 cells, the aforementioned loss of sensitivity to contact inhibition and anchorage independent growth have been confirmed, thus clearly demonstrating that the HOXB13 gene functions as an oncogene. In many cases, since a tumor forms at the injection site when NIH3T3 cells excessively expressing an oncogene are injected into nude mice, cell lines excessively expressing the HOXB13 gene can also be used in tumorigenesis experiments.

Namely, cancer cells can be produced by using the HOXB13 gene. Examples of methods of producing cancer cells include cancer cell production methods containing the following steps 1) and 2).
1) a step of transforming cells using a polynucleotide described in any one of (1) to (5) below:
   (1) a polynucleotide composed of the nucleotide sequence containing nucleotide numbers 177 to 1031 of SEQ ID NO: 1 of the Sequence Listing,
   (2) a polynucleotide composed of the nucleotide sequence containing nucleotide numbers 131 to 985 of SEQ ID NO: 1 of the Sequence Listing,
   (3) a polynucleotide composed of the nucleotide sequence containing nucleotide numbers 55 to 909 of SEQ ID NO: 3 of the Sequence Listing,
   (4) a polynucleotide composed of the nucleotide sequence containing nucleotide numbers 87 to 941 of SEQ ID NO: 5 of the Sequence Listing,
   (5) a polynucleotide that hybridizes under stringent conditions with a polynucleotide composed of a nucleotide sequence complementary to a polynucleotide described in any one of (1) to (4) above, and is composed of a nucleotide sequence that encodes a protein having an biological activity substantially identical to HOXB13 and
2) a step of selecting a cell line in which a change in cell growth ability has occurred as a result of step 1)

In the cell transformation of the aforementioned step 1), cancer cells can be developed in an animal individual by transforming cells within the animal individual, or cells can be cancerated by transforming cultured cells. Cell transformation can be carried out using an animal or cultured cells according to, for example, the method described in the aforementioned section entitled “2. Expression of the HOXB13 Gene”.

The change in cell growth ability in step 2) refers to the loss of sensitivity to contact inhibition with respect to cells or the demonstration of anchorage independent growth by the transformed cells. Although changes in cell growth ability can also be confirmed by observing a remarkable increase in the number of foci as determined by the focus formation test in cells that have been transformed in comparison with cells that have not been transformed, observing a remarkable increase in the number of colonies in a colony formation test, and/or observing a remarkable increase in the diameter of spheroids in a spheroid growth test as indicated below, methods of confirming changes in cell growth ability are not limited to these methods provided they are able to investigate changes in cell growth ability. Cell lines in which changes in cell growth ability have occurred can be selected as cancer cells.

Furthermore, cell lines that excessively express HOXB13 have one or more of the properties indicated in (1) to (3) below.

(1) Focus Formation

Normal fibroblasts such as NIH3T3 normally grow without overlapping even during dense growth, and stop growing when a single layer is formed. On the other hand, transformed cells and cancer cells lose their sensitivity to contact inhibition, and are able to continue to grow while overlapping, resulting in the formation of cell populations layered in multiple layers that have the characteristic of forming foci in which highly dense cell masses are present in the spread of the single layer of cells (Ryo Yokota, Tadashi Yamamoto, ed., Bio Manual UP Series, Cancer Research Protocol, Yodosha Co., Ltd., p. 168-174 (Oct. 15, 1995). Since this phenomenon is observed, for example, in the case of a cancer virus infecting cultured cells, in addition to the case of being used to quantify cancer viruses (Fundamental Techniques in Virology, Academic Press (1969), p. 198-211), since it can also be observed by transfecting cells with a DNA fragment containing an oncogene derived from viruses or cells, it is used for screening oncogenes and cancer inhibitory genes.

After culturing a cell line that has excessively expressed the HOXB13 gene and a cell line in which it has not been excessively expressed using ordinary methods, the culture medium is replaced with fresh medium followed by dispensing into a multi-well plate (for example, a 96-well plate, Corning Coaster 3598), culturing for a predetermined amount of time and measuring the number of foci in each well. After tabulating the number of foci per well, the results are subjected to statistical processing to calculate the mean number of foci per well and standard deviation.

NIH3T3 cells that have excessively expressed the HOXB13 gene are observed to demonstrate remarkable increases in the number of foci as compared with cells that have not excessively expressed the HOXB13 gene.

(2) Colony Formation Test

With the exception of blood cells, normal cells are known to be unable to grow if placed in a state in which there is no anchor to adhere. In contrast, transformed cells and cancer cells have the characteristic of being able to grow even in the absence of such an anchor. This characteristic change can easily be investigated by the formation of growing colonies in soft agar medium (Ryo Yokota, Tadashi Yamamoto, ed., Bio Manual UP Series, Cancer Research Protocol, Yodosha Co., Ltd., p. 168-174 (Oct. 15, 1995).

After culturing a cell line in which the HOXB13 gene has been excessively expressed and a cell line in which the HOXB13 gene has not been excessively expressed by ordinary methods and replacing the medium with fresh medium, the cells are suspended in soft agar medium (for example, RPMI1640 containing 0.33% Bactoagar (Difco) and 20% FCS) and then layered on agar medium (for example, RPMI1640 containing 0.66% Bactoagar and 20% FCS). The cells are then cultured under ordinary conditions (for example, 37° C. and 5% CO₂) followed by measuring the number of growing colonies that have formed in the soft agar. In the case of culturing using a multi-well plate (for example, a 12-well plate, Corning Coaster 3512), the number of colonies per well is measured after culturing for a predetermined amount of time. After tabulating the number of colonies per well, the results are subjected to statistical processing to calculate the mean number of colonies per well and standard deviation.

NIH3T3 cell line that has excessively expressed the HOXB13 gene is observed to demonstrate remarkable increases in the number of colonies as compared with cells that have not excessively expressed the HOXB13 gene.

(3) Spheroid Growth Test

The characteristic of anchorage-independent growth exhibited by transformed cells and cancer cells can also be easily evaluated by culturing in a spheroid state. Spheroids are aggregates consisting of large numbers of cells have gathered together, and can be easily formed by culturing on a culture plate in which cell adhesion has been suppressed to an extremely low level. Culturing in the spheroid state differs from ordinary single layer culturing in that it resembles culturing in the anchorage-independent state similar to that indicated in the aforementioned section entitled “(2) Colony Formation Test”, and function can be observed under conditions approximating those in the body (SUMILON Physicochemical Instrument General Catalog, Sumitomo Bakelite).

After culturing a cell line in which the HOXB13 gene has been excessively expressed and a cell line in which the HOXB13 gene has not been excessively expressed by ordinary methods and replacing the medium with fresh medium, the cells are dispensed onto a non-cell-adhering multi-well plate (for example, Spheroid 96U, Sumitomo Bakelite MS-0096S). The cells are cultured under ordinary conditions (for example, 37° C. and 5% CO₂) followed by measuring the size of the spheroids that form on the plate after the passage of a predetermined amount of time after the start of culturing. Spheroid size can be measured microscopically using, for example, an eyepiece grid micrometer (Sankei, S-6). Spheroid diameter can be used as an indicator of spheroid size.

NIH3T3 cells that have excessively expressed the HOXB13 gene are observed to demonstrate remarkable increases in spheroid diameter as compared with cells that have not excessively expressed the HOXB13 gene.

4. Function of HOXB13

Physiological functions of HOXB13 produced by genetic engineering methods utilizing the HOXB13 gene can be studied in expression experiments using virus.

For example, the HOXB13 gene is amplified by PCR-method, and the amplified gene is incorporated into an expression vector for adenovirus or a retrovirus vector. Commercially available kits are also used at this time (e.g. Adenovirus Expression Vector Kit (TAKARA Shuzo) and Retro-X System (Clontech)) . Virus thus obtained is inoculated into, for example, mice (ex. Balb/c mouse (CLEA Japan, Inc.) via vena caudalis and an adenovirus without the HOXB13 gene, as control, and inoculated then levels of ALT (alanine aminotransferase) and tumor marker gene expression in blood are measured. Also, various tissues are removed from the mouse and their conditions are examined. In the examination, if some tissue specific promoters (e.g., rat probasin promoter (Greenberg et al., Mol. Endocrinol. (1994) p. 230-239), mouse breast cancer virus promoter (Wynshaw-Boris, Cancer Handbook 2 (2002), Nature Publishing Group, London, p. 891-902 and the like) are used, the tissues specifically expressing corresponding genes are removed and examined for their conditions. By comparing with control level, physiological functions of HOXB13 can be understood.

5. Method for Treating and/or Preventing of Cancer

A nucleotide sequence complementary to a nucleotide sequence containing nucleotide numbers 1 to 1316 of SEQ ID NO: 1, nucleotide numbers 1 to 1270 of SEQ ID NO: 3, nucleotide numbers 1 to 1026 of SEQ ID NO: 5, or nucleotide numbers 1 to 1356 of SEQ ID NO: 7 of the Sequence Listing, preferably nucleotide numbers 177 to 1031 of SEQ ID NO: 1, nucleotide numbers 131 to 985 of SEQ ID NO: 3, nucleotide numbers 55 to 909 of SEQ ID NO: 5, or nucleotide numbers 87 to 941 of SEQ ID NO: 7 of the Sequence Listing or a nucleotide sequence complementary to a partial sequence of the sequences can be used for an antisense treatment. An antisense molecule can be used as DNA normally comprised of 15 to 30 mer, or a stable DNA derivative such as phosphorothioate, methyl phosphonate morpholino derivative, a stable RNA derivative such as 2′-O-alkyl RNA complementary to a partial sequence of nucleotide sequence indicated with nucleotide numbers 1 to 1316 of SEQ ID NO: 1, nucleotide numbers 1 to 1270 of SEQ ID NO: 3, nucleotide numbers 1 to 1026 of SEQ ID NO: 5, or nucleotide numbers 1 to 1356 of SEQ ID NO: 7 of the Sequence Listing, preferably nucleotide numbers 177 to 1031 of SEQ ID NO: 1, nucleotide numbers 131 to 985 of SEQ ID NO: 3, nucleotide numbers 55 to 909 of SEQ ID NO: 5, or nucleotide numbers 87 to 941 of SEQ ID NO: 7 of the Sequence Listing.

A siRNA for HOXB13, for example, the nucleotide sequence containing the nucleotide numbers 177 to 1031 of SEQ ID NO: 1, the nucleotide numbers 131 to 985 of SEQ ID NO: 3, the nucleotide numbers 55 to 909 of SEQ ID NO: 5, or the nucleotide numbers 87 to 941 of SEQ ID NO: 7 of the Sequence Listing can be used for the treatment for cancer, for example, human prostate cancer, human lung cancer, human breast cancer, human stomach cancer, human colon cancer, human malignant melanoma, human pancreatic cancer, preferably, prostate cancer.

A siRNA selected from the group consisting of the following 1) to 5) can be used for the treatment for cancer, for example, human prostate cancer, human lung cancer, human breast cancer, human stomach cancer, human colon cancer, human malignant melanoma, human pancreatic cancer, preferably, prostate cancer:
1) a siRNA comprising the combination of an oligonucleotide composed of the nucleotide sequence of SEQ ID NO: 13 of the Sequence Listing, and an oligonucleotide composed of the nucleotide sequence of SEQ ID NO: 14 of the Sequence Listing;
2) a siRNA comprising the combination of an oligonucleotide composed of the nucleotide sequence of SEQ ID NO: 15 of the Sequence Listing, and an oligonucleotide composed of the nucleotide sequence of SEQ ID NO: 16 of the Sequence Listing;
3) a siRNA comprising the combination of an oligonucleotide composed of the nucleotide sequence of SEQ ID NO: 17 of the Sequence Listing, and an oligonucleotide composed of the nucleotide sequence of SEQ ID NO: 18 of the Sequence Listing;
4) a siRNA comprising the combination of an oligonucleotide composed of the nucleotide sequence of SEQ ID NO: 27 of the Sequence Listing, and an oligonucleotide composed of the nucleotide sequence of SEQ ID NO: 28 of the Sequence Listing;
5) a siRNA comprising the combination of an oligonucleotide composed of the nucleotide sequence of SEQ ID NO: 29 of the Sequence Listing, and an oligonucleotide composed of the nucleotide sequence of SEQ ID NO: 30 of the Sequence Listing.

Such antisense molecule and/or siRNA can be introduced into cells by using a technology well known in the art such as microinjection, liposome-encapsulation and expression of vector having the corresponding antisense sequence. A siRNA can be introduced after mixing with athercollagen and cationic liposomes (Nucleic Acids Research, 2004, Vol. 32, e. 109: online publication). Such antisense therapy is useful in treatment or prevention of diseases induced by an over-increase in activity of a protein that is coded by a sequence of nucleotide numbers 177 to 1031 of SEQ ID NO: 1, nucleotide numbers 131 to 985 of SEQ ID NO: 3, nucleotide numbers 55 to 909 of SEQ ID NO: 5 or nucleotide numbers 87 to 941 of SEQ ID NO: 7.

The composition useful as a pharmaceutical preparation containing the antisense oligonucleotide and/or siRNA described above may be produced by well known methods such as mixing with a pharmaceutical acceptable carrier. Examples of such carriers and production methods are described in Applied Antisense oligonucleotide Technology (1998 Wiley-Liss, Inc.). A pharmaceutical preparation containing antisense oligonucleotides and/or siRNA can be administered orally as it is or by mixing with an appropriate pharmacologically acceptable carrier, such as an excipient or a diluent in the form of tablets, capsules, granule, powder or syrup, or can be administered parentally by injection, suppository, patch or an external preparation. These pharamaceutical preparations can be produced by a well known method using an additive including an excipient (e.g., organic strain excipients including sugar derivatives such as lactose, sucrose, glucose, mannitol and sorbitol; starch derivatives such as corn starch, potato starch, a starch, dextrin; a cellulose derivative such as crystalline cellulose; arabic gum; dextran; and pullulan; and inorganic excipients including silicate derivatives such as light anhydrous silicic acid, synthetic aluminum silicate, calcium silicate, and magnesium aluminometasilicate, a phosphate such as calcium hydrogen phosphate; carbonate such as calcium carbonate, and sulfate such as calcium sulfate), a lubricant (e.g., metal stearate such as stearic acid, calcium stearate, and magnesium stearate; talc; colloidal silica; wax such as bead wax, and spermaceti; boric acid; adipic acid; a sulfate such as sodium sulfate; glycol; fumaric acid; sodium benzoate; DL leucine; lauryl sulfate such as sodium lauryl sulfate, and lauryl magnesium sulfate; silicic acid such as silicic acid anhydride, and silicic acid hydrate; and the above-mentioned starch derivatives), a binding agent (e.g., hydroxypropyl cellulose, hydroxypropyl methyl cellulose, polyvinylpyrrolidone, macrogol, and compounds similar to the above-mentioned excipients), a disintegrating agent (e.g., cellulose derivatives, such as low substitution degree hydroxypropylcellulose, carboxymethyl cellulose, carboxymethyl cellulose calcium, internally cross-linked sodium carboxymethyl cellulose; chemically modified starchcellulose such as carboxymethyl starch flour, carboxymethyl starch flour sodium, cross-linked polyvinylpyrrolidone), an emulsifier (e.g., colloidal clay such as bentonite, and beegum; a metalhydroxide such as magnesium hydroxide, and aluminium hydroxide; an anionic surfactant such as sodium lauryl sulfate, and calcium stearate; a cationic surfactant such as benzalkonium chloride; a nonionic surfactant such as polyoxyethylene alkyl ether, polyoxyethylene sorbitan fatty acid ester, and sucrose fatty acid ester), a stabilizer (paraoxybenzoic acids such as methylparaben, and propylparaben; alcohols such as chlorobutanol, benzyl alcohol, phenylethyl alcohol; benzalkonium chloride; phenols such as phenol and cresol; thimerosal; dehydroacetic acid; sorbic acid), flavoring agent (e.g., generally used sweeteners, acidulants, and flavors), and additives such as diluents.

In addition to the method above, a method using a colloid dispersion system can be used as a method for introducing compounds of the present invention into patients. The colloid dispersion system is expected to be a system effective in enhancement of compound stability in vivo and for efficient transport of the compound to a specific organ, tissue or cell. Any conventional colloid dispersion system may be used with no limitation. It includes a colloid dispersion system based on a lipid involving a macromolecular complex, a nano-capsule, a microsphere, beads, an oil-in-water emulsion, a micell, a mixed micell and a liposome, and liposomes, artificial vesicles, which are effective in transporting compounds to a specific organ, tissue or cell, are preferable (Mannino et al., Biotechniques, 1988, 6, 682; Blume and Cevc, Biochem. et Biophys. Acta, 1990, 1029, 91; Lappalainen et al., Antiviral Res., 1994, 23, 119; Chonn and Cullis, Current Op. Biotech., 1995, 6, 698).

A mono-membrane liposome within a range of 0.2-0.4 um can encapsulate a significant rate of an aqueous buffer containing a macromolecule. Compounds are encapsulated by this aqueous inner membrane and transported into brain cells keeping their biological activities (Fraley et al., Trends Biochem. Sci., 1981, 6, 77). A liposome is a complex composed of a lipid, particularly phospholipid, especially a phospholipid having a high phase transition temperature, and one or more steroids, particularly cholesterol. Examples of a lipid useful for liposome production include a phosphatidyl compound such as phosphatidylglycerol, phosphatidylcholine, phosphatidylserine, sphingolipid, phosphatidylethanolamine, cerebroside, and ganglioside. A particularly useful lipid is diacylphosphatidylglycerol, of which the lipid-part contains 14-18 carbon atoms, particularly 16-18 carbon atoms and are saturated (i.e., lack a double bond in a 14-18 carbon atom chain). Examples of phospholipids include phosphatidylcholine, dipalmitoylphosphatidylcholine and distearoylphosphatidylcholine.

Targeting of colloid dispersion system containing liposome may be either a passive or active one. Passive targeting can be achieved by utilizing a tendency inherently provided in liposome, distributing into the reticuloendothelial cell in organs having sinusoid. On the other hand, an example of active targeting is, for example, a method to attach aimed ligands such as a virus protein coat (Morishita et al., Proc. Natl. Acad. Sci. (U.S.A.), 1993, 90, 8474), a monoclonal antibody (or appropriate fragment thereof), a sugar, a glycolipid or a protein (or an appropriate oligopeptide fragment thereof) to a liposome, or a method for modifying a liposome by changing its composition and thereby it is possible to distribute it to organs of cells where the macromolecule in question is originally not localized. The targeting surface of the colloid dispersion system can be modified by various methods. In a liposome targeting delivery system, a lipid group is introduced into a lipid bilayer of a liposome to support a targeting ligand at close association with the lipid bilayer. Various linkers can be used in binding a lipid chain to a targeting ligand. Targeting ligand predominantly found on cell to which delivery of the oligonucleotide of the invention is desired, which binds to a specific cell surface molecule, may be, for example, (1) a hormone, growth factor or a suitable fragment thereof which binds to a specific cell receptor expressed predominantly on a cell to which the delivery is desired, or (2) a polyclonal antibody, a monoclonal antibody, or an appropriate fragment thereof (for example, Fab; F(ab′)2) which binds specifically to an antigenic epitope predominantly found on a target cell. Also, two or more biological active agents can be combined within one liposome and administered. Agents enhancing intracellular stability and/or targeting efficiency can be added to the colloid dispersion system.

The dosage varies depending on the condition, age, and the like of the patient, for example, a mammal or a warm-blooded animal, e.g., a human. For internal use, a unit dose (daily dose) range between a minimum dose of 0.01 mg/kg to 1 g/kg of body weight can be administered. For injection use, a unit dose (daily dose) ranges between 0.001 mg/kg to 0.5 g/kg and can be administered subcutaneously, intraveneously or intramuscularly. For example, for a human, for internal use, a unit dose (daily dose) range between a minimum dose of 1 mg (suitably, 30 mg) and a maximum dose of 2000 mg (suitably, 1500 mg) can be administered; for injection use, a unit dose (daily dose) which ranges between a lower dose of 0.1 mg (suitably, 5 mg) and a maximum dose of 1000 mg (suitably, 500 mg) can be administered via subcutaneous, intramuscular or intravenous injection.

### EXAMPLES

The present invention will now be described with reference to the following examples and it should be appreciated that the invention is not limited by these examples in any way. All procedures for genetic manipulation used in the following examples were carried out according to the methods described in “Molecular Cloning, Sambrook, J., Fritsch, E. F. and Maniatis, T, Cold Spring Harbor Laboratory Press, 1989” or instructions attached to commercially available reagents or kits used, if not specified otherwise.

### Example 1 Acquisition of Human HOXB13 cDNA Clone

a) Total RNA Isolation

A human prostate cancer cell line, LNCaP (American Tissue Culture Collection ATCC No.: CRL-1740) was cultured in 75 cm² tissue culture flask (Sumitomo Bakelite) with RPMI 1640 medium (Asahi Techno Glass) containing 10% fetal calf serum (FCS). The cell was cultured in a condition of 5% CO₂ and 37° C. with taking care that the cell did not become confluent. The cell was detached and collected from the flask with trypsin-EDTA solution (Sigma) during its exponential growth period. Its aliquot was transferred to a fresh culture flask for subculture. Total RNA was isolated from a human prostate cancer cell line, LNCaP, which is collected during exponential growth, by using TRIzol (Invitrogen) according to the protocol associated therewith.

b) First Strand cDNA Synthesis

First strand cDNAs were synthesized from the total RNA isolated in the example 1a by using Omniscript Reverse Transcriptase (Qiagen) according to the protocol associated therewith. The reaction was carried out in the volume of 20 μl.

c) PCR Reaction

Following primers were synthesized as primers for HOXB13 cDNA amplification:

5′-caccatggagcccggcaattatgcca-3′
(Primer 1: SEQ ID NO: 9)
and
5′-ttaaggggtagcgctgttctt-3′
(Primer 2: SEQ ID NO: 10)

Primer 1 is an oligonucleotide constructed by adding 4 bases, CACC, as a Kozak sequence to be upstream of the initiation codon of the HOXB13 gene. Its sequence corresponds to a sequence of nucleotide numbers 395 to 410 of SEQ ID NO: 1 plus the four bases (CACC) at the 5′ site. When the oligonucleotide is introduced into the cloning vector, the pENTR/SD/D-TOPO, CACC sequence will form a complementary strand to the 3′-teminal sequence of the vector and ensure directional gene introduction. Primer 2 is an oligonucleotide complementary to the sequence of nucleotide numbers 1498 to 1519 of SEQ ID NO: 1.

A PCR reaction was performed by using ProofStart DNA Polymerase (Qiagen) according to the protocol associated therewith. Specifically, 0.5 μl of each of synthesized primer 1 and primer 2, 5 μl of 10× ProofStart PCR Buffer, 1.5 μl of 10 mM dNTP Mix, 2 μl of ProofStart DNA Polymerase, 10 μl of 5× Q-Solution, and 29.5 μl of sterilized purified water was added to 1 μl of the first strand cDNA obtained to make 50 μl of a PCR reaction mixture. A PCR reaction was performed on a GeneAmp PCR System 9700 (Applied Biosystems) . After treatment at 95° C. for 5 minutes, a cycle of 95° C. for 30 seconds, 57° C. for 30 seconds and 72° C. for 4 minutes was repeated for 35 cycles, followed by treatment at 72° C. for 20 min, and then stored at 4° C. The aimed cDNA was obtained by subjecting the PCR product onto 1% agarose gel electrophoresis, confirming amplification of HOXB13 cDNA (855bp) and then isolating the cDNA from the agarose gel by using a QIAquick Gel Extraction Kit (Quiagen) according to the protocol associated therewith. The concentration of the purified HOXB13 cDNA was measured by a spectrophotometer (Gene Spec I: Hitachi Instrument Service).

d) Cloning of HOXB13 cDNA into pENTR/SD/D-TOPO Vector

HOXB13 cDNA obtained in Example 1c was cloned into a pENTR/SD/D-TOPO vector by use of pENTR DIrectional TOPO Cloning Kits (Invitrogen). HOXB13 cDNA was mixed with topoisomerase bound pENTR/SD/D-TOPO vector in the reaction buffer that is attached to the kit and incubated at room temperature for 5 minutes. E. coli cell, OneShot TOP10 Chemically Competent E. coli (Invitrogen), was transformed with the reaction product obtained and incubated in LB agar medium containing 50 μg/ml kanamycin. After incubation, plasmid DNAs were isolated by selecting E. coli colonies that show kanamycin resistance and growth, culturing them in 0.3 ml of liquid TBG medium containing 50 μg/ml of kanamycin at 37° C. overnight and isolating plasmid DNAs with the help of BIO ROBOT 9600 (Qiagen). Isolated plasmid DNAs were subjected to ABI PRISM 3700 DNA Analyzer (Applied Biosystems) to analyze their nucleotide sequences, and it was confirmed that cDNA containing the open reading frame of the nucleotide sequence (SEQ ID NO: 1) specified in GeneBank accession No. U81599 was integrated into a pENTR/SD/D-TOPO vector.

e) Cloning into a Retrovirus Vector, pLNCX

Reading Frame Cassette A, a DNA fragment contained in GATEWAY Vector Conversion System (Invitrogen) was inserted at the HpaI site of pLNCX, a retrovirus vector contained in RetroXpress System (Clonetech), in the forward direction related to the CMV promoter in the plasmid to prepare a modified vector, pLNCX-GW. LR CLONASE Enzyme Mix (Invitrogen) was used according to the protocol associated therewith to give a recombinant vector in which HOXB13 cDNA cloned in the pENTR/SD/D-TOPO vector was transported to the pLNCX-GW. An E. coli cell, OneShot TOP10 Chemically Competent E. coli was transformed with this recombinant vector and cultured in LB agar medium containing 50 μg/ml of ampicillin. Growing ampicillin resistant E. coli colonies were selected and cultured in 0.3 ml of liquid TBG media containing 50 μg/ml of ampicillin at 37° C. overnight, and then plasmid DNAs were isolated with the help of BIO ROBOT 9600 (Qiagen). An E. coli cell, OneShot TOP10 Chemically Competent E. coli (Invitrogen) was transformed with the plasmid DNAs obtained and cultured in LB agar containing 50 μg/ml of ampicillin. A growing ampicillin resistant E. coli colony, i.e., colony was selected and cultured in 150 ml of liquid TBG medium containing 50 μg/ml of ampicillin at 37° C. overnight, and then the plasmid was purified from the culture medium by using Endo Free Plasmid Maxi Kits (Quiagen) according to the protocol associated therewith. Purified plasmid was named pLNCX-GW-HOXB13. Confirmation that the inserted gene in this plasmid was HOXB13 cDNA was carried out by digesting the plasmid and analyzing lengths of digested DNA fragments on agarose electrophoresis.

### Example 2 Preparation of HOXB13 Gene Expression Retrovirus and Establishment of HOXB13 Stably Expressing Cell Line

a) Cell Line and its Subculture

A packaging cell line, 293-1A1 (IMGENEX) and a mouse fibroblast cell line, NIH3T3 (American Tissue Culture Collection) were cultured in 25, 75 or 225 cm² tissue culture flask (Corning Coaster or Sumitomo Bakelite) with RPMI 1640 medium (Asahi Techno Glass) containing 10% fetal calf serum (FCS:Hyclone). The cell lines were cultured under the condition of 5% CO₂ and 37° C., taking care that the cells did not become confluent, and they were detached and collected from the flask with trypsin-EDTA solution (Sigma) during their exponential growth period. Their aliquots were transferred to fresh culture flasks and sub-cultured.

b) Preparation of a Gene Expression Retrovirus and Establishment of a Stably Expressing Cell Line

293-10A1 cell was inoculated on cell culture dish coated with type I collagen (Asahi Techno Glass), 10 cm in diameter, at a cell density of 2×10⁶ and cultured in 10 ml of RPMI 1640 medium containing 10% FCS overnight. After replacing the culture supernatant with 2 ml of fresh RPMI 1640, about 10 μg of pLNCX-GW-HOXB13 vector was introduced into the cell by using Lipofectamine 2000 (Invitrogen) according to the protocol associated therewith. After a 6 hour cultivation, 6 ml of RPMI 1640 medium containing 20% FCS was added and then the cell was cultured one night more. After that, the medium was replaced with fresh RPMI 1640 medium containing 20% of FCS and the cell was further cultured for 24 hours to produce the virus. Medium supernatant containing the virus was collected and filtrated through a 0.45 um pore-sized filter (MILLEX-HV: Millipore). A two fold-volume of fresh RPMI 1640 containing 10% FCS and Polybrene (also known as Hexadimethrine bromide)(Sigma), at a final concentration of 8 μg/ml, were added to the filtrate and mixed to prepare a virus-infecting solution. This virus-infecting solution was added to culture dishes (430293; Corning Coaster), in which 1.2×10⁶ NIH3T3 cell had been inoculated and cultured overnight, to infect the cell with the virus. This procedure was repeated every 24 hours, four times. After an additional three days cultivation, to remove uninfected cells, i.e., cells not expressing the aimed gene, the culture medium was replaced with a RPMI 1640 medium containing 500 μg/ml of Geneticin (Invitrogen) and 10% of FCS. After that, the medium was replaced with fresh medium containing Geneticin every two to three days. The cells were cultured for seven days in such a manner to establish cell lines stably expressing the aimed gene. Expression of the aimed gene in the established cell line was confirmed by RT-PCR using the following primer set, which can amplify the fragment inserted into PLNCX-GW.

5′-ccaaaatgtcgtaacaactc-3′
(Primer 3: SEQ ID NO: 11 of the Sequence Listing)
5′-gaccttgatctgaacttctc-3′
(Primer 4: SEQ ID NO: 12 of the Sequence Listing)

Primers 3 and 4 are oligonucleotides that have been designed based on the nucleotide sequence of PLNCX and can amplify the DNA fragment inserted into pLNCX.

### Example 3 Colony Formation Test

An established cell line stably expressing the HOXB13 gene and a control cell strain, genetically unmodified parent cell, were collected by subjecting them to trypsin-EDTA treatment, and washed with fresh medium twice. After that, 50,000 cells were warmed at about 38-39° C. per one well, suspended in 1 ml of RPMI 1640 medium containing 0.33% Bactoagar in the liquid state and 20% of FCS, and immediately poured into a 12 well plate (3512:Corning Coaster) in which a RPMI 1640 medium containing 0.66% of Bactoagar (Difco) and 20% of FCS had been dispensed and solidified. The three same wells were prepared for each cell. Plates were left at room temperature for 30 minutes so as to solidify the Bactoagar completely, and incubated under the condition of 5% CO₂ and 37° C. for 17 days. Then, the number of colonies growing in the soft agar was measured.

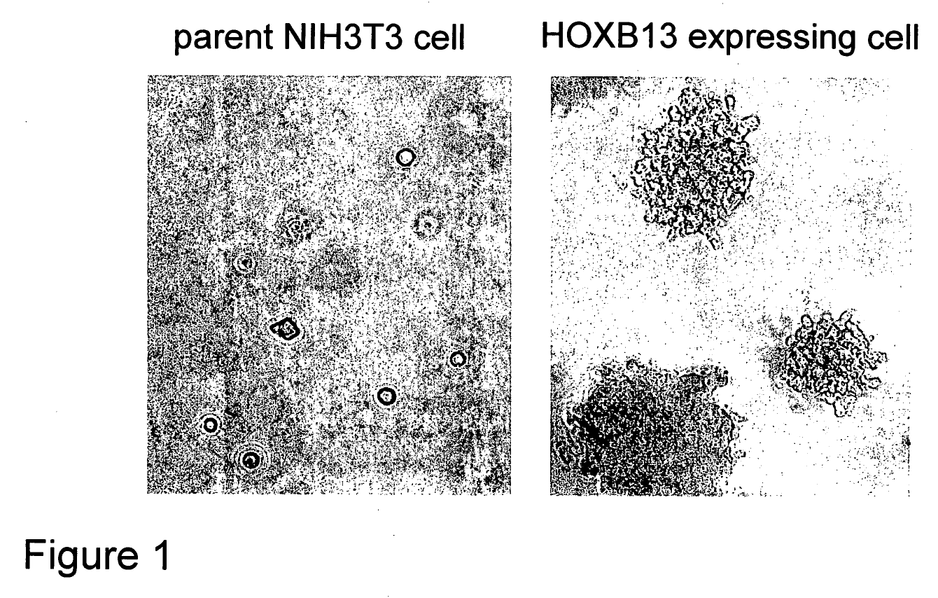

The image observed by a microscope (Nikon, DIAPHOTO300) after culture is shown in FIG. 1. While only 2.0±2.0 (average ± standard deviation) colonies were observed in the control cell, parent NIH3T3 cell line, but 1999.3±239.2 (average ± standard deviation) colonies were detected in the HOXB13 expressing cell line (Table 1), strongly suggesting that HOXB1 has a colony inducing activity.

TABLE 1

| | | | | | standard |
|---|---|---|---|---|---|
| Cell | colony number | | | average | variation |
| parent cell | 0 | 4 | 2 | 2.0 | 2.0 |
| HIXB13 expressing cell | 2,244 | 1,766 | 1,988 | 1,999.3 | 239.2 |

### Example 4 Spheroid Growth Test

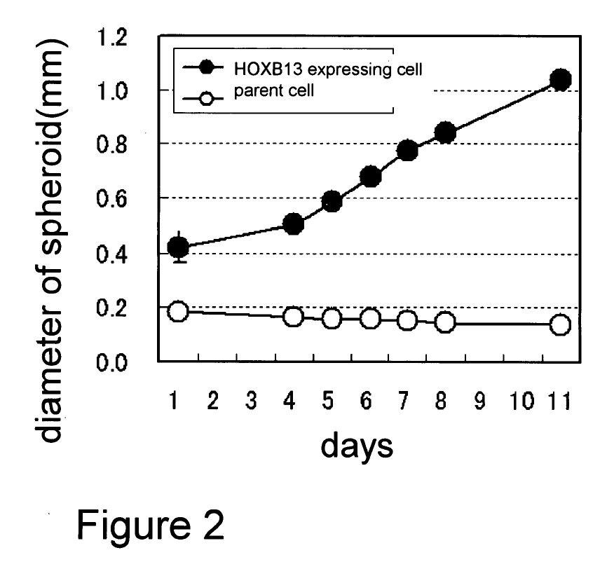

An established cell line stably expressing HOXB13 gene and an original NIH3T3 cell, not genetically modified parent cell, were subjected to a trypsin-EDTA treatment and collected. After washing with a fresh medium twice, the cell suspension was prepared with a fresh culture medium and 200 μl of the suspension, 1,000 cells, were dispensed into a well of a non-cell adhesive 96 well plate (Spheroid 96U, Sumitomo Bakelite, MS-0096S). The three same wells were prepared for each cell. Cells were cultured under the condition of 5% CO₂ and 37° C., and diameters of spherical cell aggregates (Spheroid) formed on the plates were measured with a microscope (Nikon, DIAPHOTO300) and an ocular micrometer (Sankei: S-6) on the 1st, 4th, 5th, 6th, 7th, 8th and 11th days after the start of the culture. These results are shown in Table 2 and FIG. 2. In the control, the parent cell line, the diameter of spheroids kept unchanged and no spheroid growth was observed in the non-cell adhesive plate, but on the contrary, a prominent increase in the spheroid diameter was continuously observed for the HOXB13 expressing cell. This strongly suggests that HOXB13 induces spheroid growth of NIH3T3 cell.

TABLE 2

| | diameter of spheroid (mm) | | | | | | | | | | | |
|---|---|---|---|---|---|---|---|---|---|---|---|---|
| cell | Day 1 | | | Day 4 | | | Day 5 | | | Day 6 | | |
| parent cell | 0.18 | 0.19 | 0.18 | 0.15 | 0.17 | 0.16 | 0.15 | 0.16 | 0.16 | 0.16 | 0.15 | 0.15 |
| OXB13 expressing | 0.48 | 0.37 | 0.41 | 0.49 | 0.50 | 0.51 | 0.59 | 0.60 | 0.58 | 0.68 | 0.68 | 0.67 |
| cell | | | | | | | | | | | | |

| | diameter of spheroid (mm) | | | | | | | | |
|---|---|---|---|---|---|---|---|---|---|
| | Day 7 | | | Day 8 | | | Day 11 | | |
| parent cell | 0.14 | 0.16 | 0.15 | 0.14 | 0.15 | 0.15 | 0.14 | 0.14 | 0.14 |
| HOXB13 expressing | 0.78 | 0.77 | 0.78 | 0.84 | 0.84 | 0.85 | 1.05 | 1.03 | 1.03 |
| cell | | | | | | | | | |

### Example 5 Growth Inhibition Test Using siRNA on Human Prostate Cancer Cell Line, LNCap

(1) Cell Line and its Subculture

Human prostate cancer cell line LNCaP (American Tissue Culture Collection ATCC No.: CRL-1740) was cultured in 75 cm² tissue culture flask (Sumitomo Bakelite) with RPMI 1640 medium (Asahi Techno Glass) containing 10% fetal calf serum (FCS: Hyclone) under the condition of 5% CO₂ and 37° C., taking care that the culture did not become confluent. The cell was detached and collected from the flask with trypsin-EDTA solution (Sigma) during its exponential growth period. Its aliquot was transferred to a fresh culture flask and sub-cultured.

(2) Growth Suppression Test and Gene Expression Suppression Test using siRNA on a Human Prostate Cancer Cell Line, LNCap

Forty thousand cells of a human prostate cancer cell line LNCaP were dispensed into a poly-D-lysine coated tissue culture 24 well dish (Poly-D-Lysine Cellware 24-Well Plate: Beckton Dickinson) and cultured in 0.4 ml of RPMI 1640 medium containing 10% of FCS overnight. After replacing the medium supernatant with 0.4 ml of fresh RPMI 1640 medium, the medium was further changed to 0.2 ml of RPMI 1640 medium containing 200 nM siRNA complementary to any gene and 1.2% of DMRIE-C Reagent (Invitrogen). The cells were cultured for 4 hours in order to introduce siRNA into the cells.

In this case, the nucleotide sequence of siRNA No. 1, used for HOXB13 is homologous with the sequence of the nucleotide numbers 125 to 143 of SEQ ID NO: 7 of the Sequence Listing and consist of the combination of the following sequences:

5′-ggauaucgaaggcuugcugtt-3′
(SEQ ID NO: 13 of the Sequence Listing)
and,
5′-cagcaagccuucgauaucctt-3′
(SEQ ID NO: 14 of the Sequence Listing).

The nucleotide sequence of siRNA No. 2 for HOXB13 is homologous with the sequence of the nucleotide numbers 803 to 821 of SEQ ID NO: 7 of the Sequence Listing and consists of the combination of the following sequences

5′-guucaucaccaaggacaagtt-3′
(SEQ ID NO: 15 of the Sequence Listing)
and,
5′-cuuguccuuggugaugaactt-3′
(SEQ ID NO: 16 of the Sequence Listing).

The nucleotide sequences of siRNA No. 3 for HOXB13 is homologous with the sequence of the nucleotide numbers 916 to 934 of SEQ ID NO: 7 of the Sequence Listing and consists of the combination of the following sequences:

5′-ggugaagaacagcgcuacctt-3′ (SEQ ID NO: 17 of the Sequence Listing) and,

5′-gguagcgcuguucuucacctt-3′ (SEQ ID NO: 18 of the Sequence Listing).

The culture medium was replaced with 0.4 ml of RPMI 1640 medium containing 10% FCS and the cell was cultured overnight. The cell was collected with trypsin-EDTA solution (Sigma), suspended in 2 ml of RPMI 1640 medium containing 10% FCS, dispensed into a 96 well plate (Corning Coaster, Catalog No. 3598 or 3917) 0.1 ml each, and cultured under the condition of 5% CO₂ and 37° C. Twenty four hours or 6 days after introducing siRNA into the cell, CellTiter-Glo™ Luminescent Cell Viability Assay (Promega) was used according to the protocol associated therewith to measure intracellular ATP level, and the ATP level was used as indication of cell number to calculate the rate of change in cell number, i.e., the cell growth rate. A siRNA against luciferase gene, which does not exist in human, was used as a negative control, a gene not suppressing cell growth, and a siRNA against Eg5 gene that is the essential gene for humans was used as a positive control, a gene suppressing cell growth.

The nucleotide sequence of the siRNA against luciferase consists of the combination of the following sequences:

5′-cguacgcggaauacuucgatt-3′
(SEQ ID NO: 19 of the Sequence Listing),
5′-ucgaaguauuccgcguacgtt-3′
(SEQ ID NO: 20 of the Sequence Listing).

In addition, the nucleotide sequence of the siRNA aganist human Eg5 consists of the combination of the following sequences:

5′-cuggaucguaagaaggcagtt-3′
(SEQ ID NO: 21 of the Sequence Listing),
5′-cugccuucuuacgauccagtt-3′
(SEQ ID NO: 22 of the Sequence Listing).

Furthermore, two days after introducing the siRNAs into the cell, a RNeasy Mini Kit (Qiagen) was used according to the protocol associated therewith to extract total RNA from the whole cell. Next, a QuantiTect SYBE Green RT-PCR Kit (Qiagen) was used according to the protocol associated therewith to measure the expression level of the aimed gene on the total RNA obtained. Specifically, the expression level of human GAPDH was also measured at the same time. Then an expression level ratio between the aimed gene and human GAPDH gene was calculated and the change rate of gene expression level was determined based on this ratio.

RT-PCR primers for HXOB13 amplification consist of the following sequences:

5′-cttttggaaggcagcatttgca-3′
(Primer 5: SEQ ID NO: 23 of the Sequence Listing),
5′-gtgatgaacttgttagccgcatact-3′
(Primer 6: SEQ ID NO: 24 of the Sequence Listing).

RT-PCR primers for human GAPDH amplification consist of the following sequences:

5′-gaaggtgaaggtcggagtc-3′
(Primer 7: SEQ ID NO: 25 of the Sequence Listing),
5′-gaagatggtgatgggatttc-3′
(Primer 8: SEQ ID NO: 26 of the Sequence Listing).

The results are shown in Table 3.

TABLE 3
Effect of siRNA for HOXB13 for the growth of human prostate
cancer cell line LNCap

| | | gene expression |
|---|---|---|
| siRNA | cell growth rate | level |
| siRNA for Luciferase | 100.0% ± 4.8% | 100% |
| siRNA for Eg5 | 25.9% ± 1.1% | ND |
| siRNA (No. 1) for HOXB13 | 61.5% ± 2.9% | 18% |
| siRNA (No. 2) for HOXB13 | 73.8% ± 5.8% | 22% |
| siRNA (No. 3) for HOXB13 | 73.6% ± 4.0% | 13% |

ND, not determined

In the case where in the initial five days (from 1st to 6th days after siRNA introduction), the cell growth rate of the human prostate cancer cell line LNCaP, which the siRNA against luciferase gene sequence is introduced, is 100%, the cell growth rates decreased to 25.9%±1.1%, 61.5%±2.9%, 73.8%±5.8% and 73.6%±4.0% (average ± standard deviation), respectively, when positive controls siRNA, i.e., siRNA against the sequence of the essential human Eg5 gene, siRNAs against HOXB13 gene sequence; the aforesaid siRNA No. 1. (SEQ ID NO: 13 +SEQ ID NO: 14), siRNA No. 2 (SEQ ID NO: 15 +SEQ ID NO: 16) or siRNA No. 3 (SEQ ID NO: 17 +SEQ ID NO: 18) is introduced into the cell. To determine gene expression levels performed 2 days after the introduction of the siRNAs, the gene expression levels were suppressed to 18%, 22% and 13%, respectively, when siRNAs against HOXB13 gene sequence; siRNA No. 1, No. 2 or No. 3 was introduced into the cell, based on the gene expression level observed on the cell which siRNA against luciferase gene sequence was introduced is 100%. It is considered that suppression of HOXB13 gene expression led to suppression of growth of human prostate cancer cell line LNCaP.

### Example 6

Growth inhibition test using siRNA on human prostate cancer cell line, LNCap and PC-3.

(1) Establishing Human Prostate Cancer Cell Line LNCap and HOXB13 Stably Expressing PC-3

Establishing human prostate cancer cell line LNCaP and HOXB13 stably expressing PC-3 cell line.

a) A cell line and its subculture packaging cell line, 293-10A1 (IMGENEX), as well as human prostate cancer cells LNCaP (American Tissue Culture Collection) and PC-3(American Tissue Culture Collection), were cultured in a 25, 75 or 225 cm² tissue culture flask (Corning Coaster or Bakelite) with RPMI 1640 medium (Asahi Techno Glass) containing 10% fetal calf serum (FCS: Hyclone).

The cell lines were cultured under the condition of 5% CO₂ and 37° C., taking care that the cells did not become confluent, and the cells were detached and collected from the flask with trypsin-EDTA solution (Sigma) during their exponential growth period. Their aliquots were transferred to fresh culture flasks and sub-cultured.

b) 293-10A1 cell was inoculated on a cell culture dish coated with type I collagen (Asahi Techno Glass), a diameter of 10 cm, at a cell density of 2×10⁶ and cultured in 10 ml of RPMI 1640 medium containing 10% FCS overnight.

After replacing the culture supernatant with 2 ml of fresh RPMI 1640 medium, about 10 μg of pLNCX-GW-HOXB13 vector was introduced into the cell by using Lipofectamine 2000 (Invitrogen) according to the protocol associated therewith.

After a 6 hour cultivation, 6 ml of RPMI 1640 medium containing 20% FCS was added and then the cell was cultured one night more.

The culture liquid was replaced with fresh RPMI 1640 medium containing 20% FCS and the cell was cultured for 24 hours more to produce the virus.

The culture supernatant containing the virus was collected and filtered through a 0.45 μm filter (MILLEX-HV: Millipore). Two-fold volume of fresh RPMI 1640 containing 10% FCS and Polybrene (also known as Hexadimethrine bromide) (Sigma), at a final concentration of 8 μg/ml, were added to the filtrate and mixed to prepare a virus-infecting solution.

This virus-infecting solution was added to culture dishes (430293: Corning Coaster), in which 1.2×10⁶ NIH3T3 cell had been inoculated and cultured overnight, to infect the cell with the virus.

This procedure was repeated every 12 hours, four times.

After culturing for three days more, to remove uninfected cells, i.e., cells not expressing the aimed gene, the culture medium was replaced with RPMI 1640 medium containing Geneticin (Invitrogen), 600 μg/ml for LNCaP cell or 200 μg/ml for PC-3 cell, and 10% FCS.

The medium was replaced with fresh medium containing Geneticin every two to three days. The cells were cultured for seven days in such manner to establish cell lines stably expressing the aimed gene. Expression of aimed gene in the established cell line was confirmed by RT-PCR using the following primer set, which can amplify the fragment inserted into pLNCX-GW:

5′-ccaaaatgtcgtaacaactc-3′
(Primer 3: SEQ ID NO: 11 of the Sequence Listing)
and
5′-gaccttgatctgaacttctc-3′
(Primer 4: SEQ ID NO: 12 of the Sequence Listing).

Primers 3 and 4 were oligonucleotides that had been designed based on the nucleotide sequence of pLNCX and could amplify the DNA fragment inserted into pLNCX.

(2) siRNA Specificity Confirmation Test using a HOXB13 Stably Expressing Cell Strain of Human Prostate Cancer Cell Lline LNCaP Each of 40,000 cells of a human prostate cancer cell line LNCaP, which stably expressed HOXB13, were dispensed into a poly-D-lysine coated tissue culture 24 well dish (Poly-D-Lysine Cellware 24-Well Plate: Beckton Dickinson) and cultured in 0.4 ml of RPMI 1640 medium containing 10% of FCS overnight. A human prostate cancer cell line LNCap+mock in which vector pLNCX was introduced was used as a control. After replacing the medium supernatant with 0.4 ml of fresh RPMI 1640 medium, the medium was further changed to 0.2 ml of RPMI 1640 medium containing 100 nM siRNA complementary to any one gene and 1.2% of DMRIE-C Reagent (Invitrogen), and the cells were then cultured for 4 hours so as to introduce siRNA into them.

siRNAs against HOXB13 used in this example are homologous with siRNA No. 1, siRNA No. 2 and siRNA No. 3 described hereinabove in Example 5; nucleotide numbers 1245 to 1263 contained in SEQ ID NO: 1 of the Sequence Listing; a siRNA No. 4 which is a combination of the following nucleotide sequences:

5′-caguggcaauaaucacgautt-3′
(SEQ ID NO: 27 of the Sequence Listing)
5′-aucgugauuauugccacugtt-3′
(SEQ ID NO: 28 of the Sequence Listing)

and the nucleotide numbers 1247 to 1265 of SEQ ID NO: 1 of the Sequence Listing,

a siRNA No. 5 which is a combination of the following nucleotide sequences:

5′-guggcaauaaucacgauaatt-3′
(SEQ ID NO: 29 of the Sequence Listing)
5′-uuaucgugauuauugccactt-3′
(SEQ ID NO: 30 of the Sequence Listing).

The culture liquid was replaced with 0.4 ml of fresh RPMI 1640 medium containing 10% FCS and then the cells were cultured overnight.

The cells were collected with trypsin-EDTA solution (Sigma), suspended into 2 ml of RPMI 1640 medium containing 10% FCS, dispensed into a 96 well plate (Corning Coaster, Catalog No. 3598 or 3917) 0.1 ml each, and cultured under the condition of 5% CO₂ and 37° C. Twenty four hours and 6 days after introducing siRNA into the cell, a CellTiter-Glo™ Luminescent Cell Viability Assay (Promega) was used according to the protocol associated therewith to measure intracellular ATP level, and the ATP level was used as an indication of cell number to calculate the rate of change in cell number, i.e., the cell growth rate.

A siRNA against luciferase gene, which does not exist in humans, was used as a negative control not suppressing cell growth, and a siRNA against Eg5 gene that is the essential gene for humans was used as a positive control suppressing cell growth.

siRNAs against luciferase and human Eg5 used in this example are the same as described in Example 5.

Furthermore, two days after introducing the siRNAs into the cell, a RNeasy Mini Kit (Qiagen) was used according to the protocol associated therewith to extract total RNA from the whole cell, and a QuantiTect SYBE Green RT-PCR Kit (Qiagen) was used according to the protocol associated therewith to measure the expression level of the aimed gene on the obtained total RNA.

Specifically, the expression level of human GAPDH was also measured at the same time and an expression level ratio between the aimed gene and human GAPDH gene was calculated, and the change rate of gene expression level was determined based on this ratio.

RT-PCR primers used in HOXB13 amplification were the primers 5 and 6 described in Example 5.

RT-PCR primers used in GAPDH amplification were the primers 7 and 8 described in Example 5.

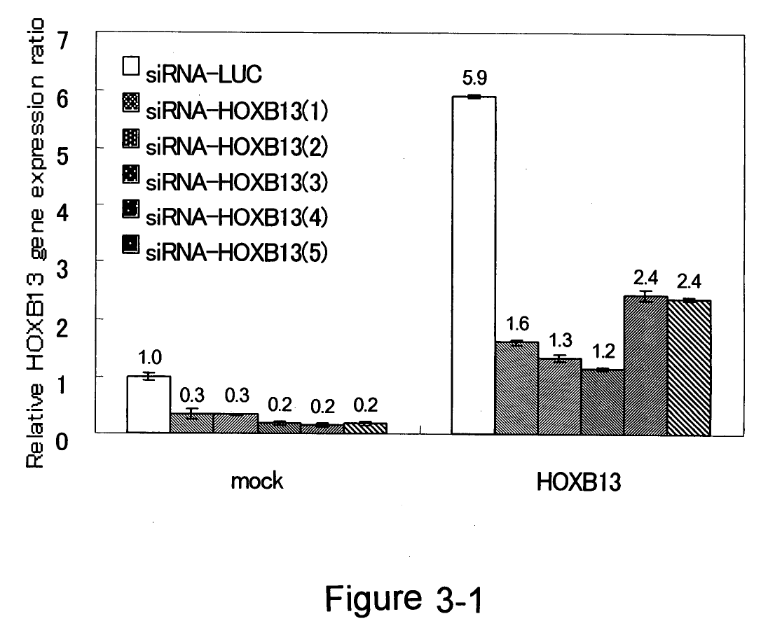

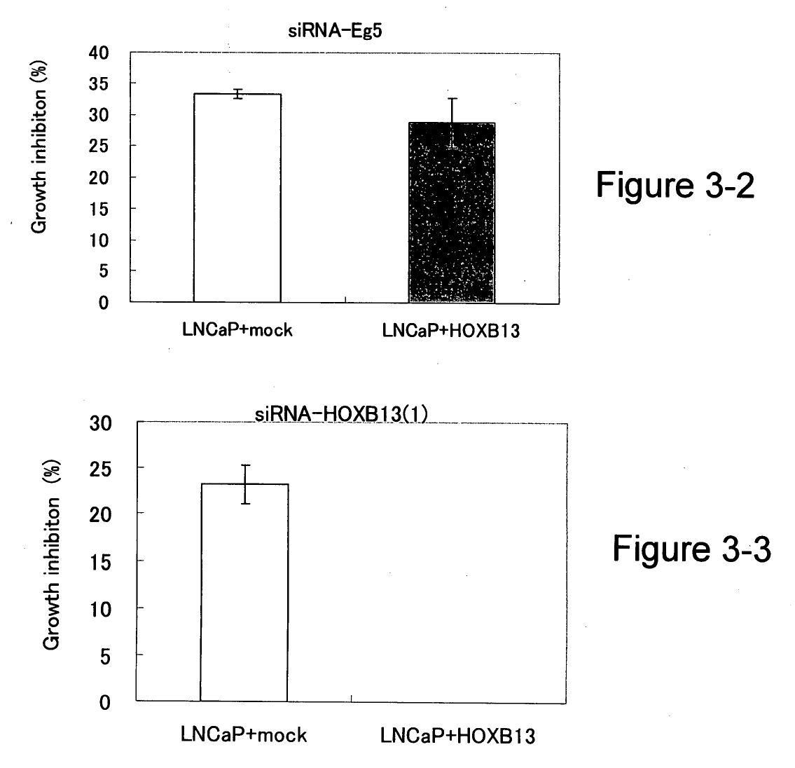

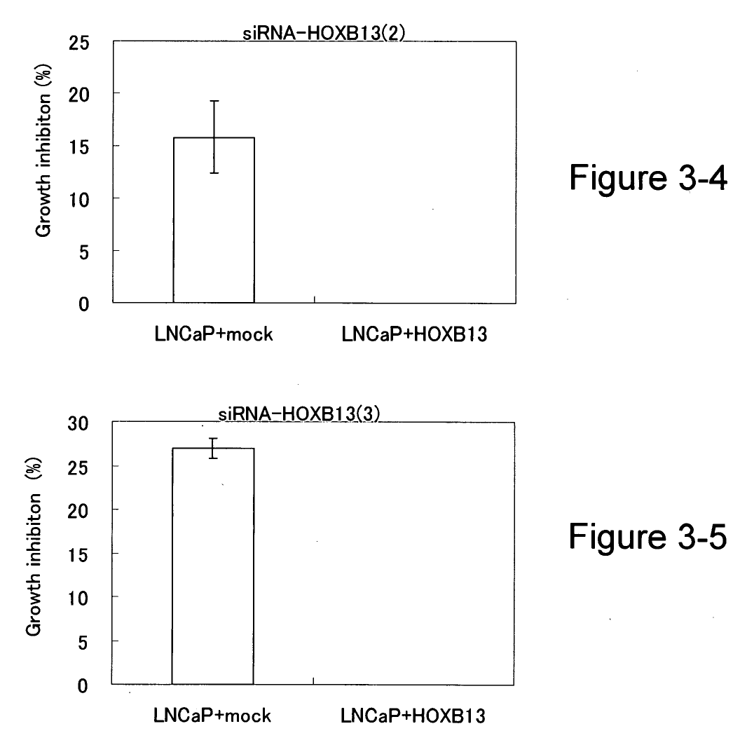

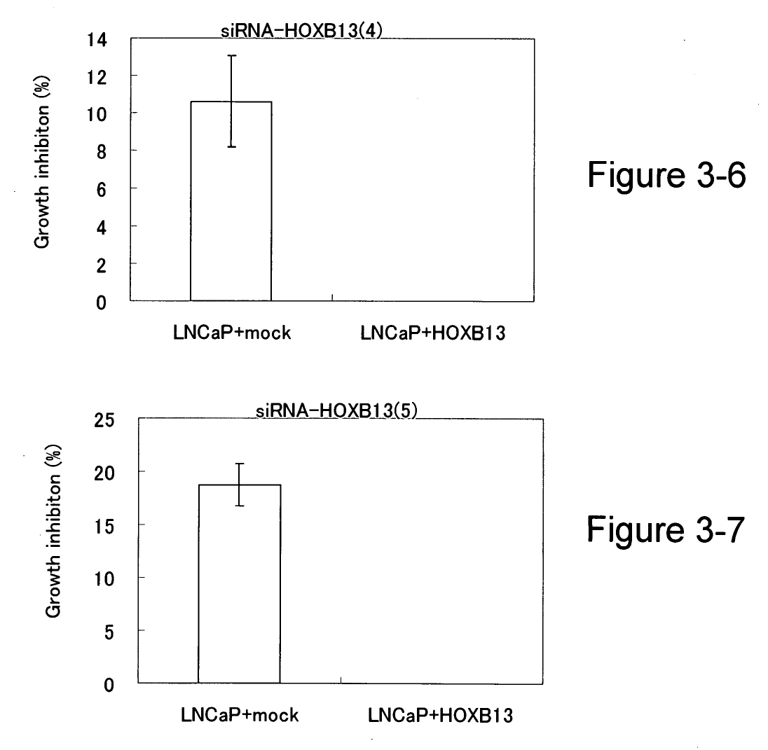

The results are shown in FIG. 3.

For measurement of gene expression level two days after siRNA introduction, it was observed that the gene expression levels were suppressed to 35%±10%, 33%±3%, 18%±3%, 15%±3% and 19%±4% (average ± standard deviation(SD)), respectively, in the cells siRNA against HOXB13 sequences No. 1, No.2, No.3, No.4 and No.5 (siRNA No. 1, siRNA No. 2, siRNA No. 3, siRNA No. 4 and siRNA No. 5, respectively,) were introduced, based on the gene expression level observed on LNCaP+mock strain where siRNA against luciferase gene sequence was introduced was 100%. In contrast thereto, for LNCaP+HOXB13 strain, a cell stably expressing HOXB13, the gene expression level was increased to 591%±127% when siRNA against luciferase gene sequence was introduced. When siRNA against HOXB13 sequence No. 1, No. 2, No. 3, No. 4, and No. 5 (siRNA No. 1, siRNA No. 2, siRNA No. 3, siRNA No. 4 and siRNA No. 5, respectively,) were introduced, the expression level of target HOXB13 gene were 161%±21%, 135%±35%, 116%±28%, 243%±65%, and 238%±34%, respectively, (average ± standard deviation(SD)). This demonstrates that the HOXB13 gene expression level of the cell exceeds that of the control strain LNCA+mock when the HOXB13 gene is introduced via retrovirus vectors, even if the expression of the gene is suppressed by siRNA.

When siRNA against Eg5 gene sequence, a human essential gene used as the positive control, and siRNAs against HOXB13 gene sequences No. 1, No. 2, No. 3, No. 4 and No. 5 (siRNA No. 1, siRNA No. 2, siRNA No. 3, siRNA No. 4 and siRNA No. 5, respectively,) were introduced into cells, growth of these cells were suppressed to 33.3%±0.8%, 23.2%±2.1%, 15.8%±3.5%, 27.0%±1.1%, 10.6%±2.4% and 18.7%±2.0%, respectively, (average ± deviation(SD)), based on the gene expression level of human prostate cancer cell line LNCaP+mock strain observed for 4 days, i.e., 1st day to 5th day after the introduction of siRNA against luciferase gene sequence being 0%.

On the other hand, in human prostate cancer cell line LNCaP+HOXB13 strain, the growth suppression rate was 28.8%±3.9% for siRNA against Eg5 gene sequence, an essential human gene, and −16.3%±3.8%, −47.8%±1.1%, −8.7%±5.5%, −44.7%±6.7% and −23.9%±4.0% (average ± standard deviation(SD))% for siRNA against HOXB13 sequences No. 1, No. 2, No. 3, No. 4 and No. 5 (siRNA No. 1, siRNA No. 2, siRNA No. 3, siRNA No. 4 and siRNA No. 5, respectively,). These results indicate that the introduction of the HOXB13 gene antagonizes the growth suppression effect observed in prostate cancer cell LNCaP when siRNA against HOXB13 gene is introduced. This suggests that the effect of HOXB13-siRNA is not nonspecific and HOXB13-siRNA exerts its growth suppression effect via suppression of HOXB13.

(3) siRNA Specificity Confirmation Test Using a HOXB13 Stably Expressing Cell Strain of Human Prostate Cancer Cell Line PC-3

Each of 40,000 cells of a human prostate cancer cell strain PC-3+HOXB13, which stably expressed HOXB13, were dispensed into a poly-D-lysine coated tissue culture 24 well dish (Poly-D-Lysine Cellware 24-Well Plate: Beckton Dickinson) and cultured in 0.4 ml of RPMI 1640 medium containing 10% of FCS overnight. A human prostate cancer cell strain PC-3+mock in which vector pLNCX was introduced was used as a control. After replacing the medium supernatant with 0.4 ml of fresh RPMI 1640 medium, the medium was further changed to 0.2 ml of RPMI 1640 medium containing 100 nM siRNA complementary to any one gene and 1.2% of DMRIE-C Reagent (Invitrogen), and the cells were then cultured for 4 hours so as to introduce siRNA into them.

The siRNAs against HOXB13 used were the same as those described in Example 6(2).

Culture medium was replaced with 0.4 ml of fresh RPMI 1640 medium containing 10% FCS and then the cells were cultured overnight.

The cells were collected with a trypsin-EDTA solution (Sigma), suspended into 2 ml of RPMI 1640 medium containing 10% FCS, dispensed into a 96 well plate (Corning Coaster, Catalog No. 3598 or 3917) 0.1 ml each, and cultured under the condition of 5% CO₂ and 37° C.

Twenty four hours or 6 days after introducing siRNA into the cell, a CellTiter-Glo™ Luminescent Cell Viability Assay (Promega) was used according to the protocol associated therewith to measure intracellular ATP level, and the ATP level was used as indication of cell number to calculate the rate of change in cell number, i.e., the cell growth rate.

The siRNA against luciferase used was the same as that described in Example 6(2). The nucleotide sequence of siRNAs against human ACTB (Homo sapiens actin, beta: GenBank Accession No. NM_{—}001101) consisting of the combination of following sequences:

5′-ugaagaucaagaucauugctt-3′
(SEQ ID NO: 31 of the Sequence Listing)
5′-gcaaugaucuugaucuucatt-3′
(SEQ ID NO: 32 of the Sequence Listing)

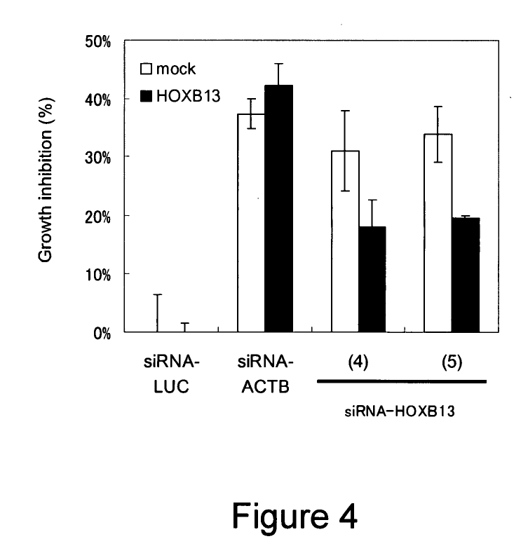

Results are shown in FIG. 4. When siRNA against ACTB gene sequence, a human essential gene used as the positive control, and siRNAs against HOXB13 gene sequences No.4 and No.5 (siRNA No. 4 and siRNA No. 5, respectively,) were introduced into cells, growth of these cells were inhibited 37.3%±2.6%, 30.9%±6.8% and 33.9%±4.8, respectively, (average ± standard deviation (SD)), based on the gene expression level of human prostate cancer cell line LNCaP+mock strain observed for 4 days , i.e., 1st day to 5th day, after the introduction of siRNA against luciferase gene sequence, being 0%. On the other hand, in human prostate cancer cell line LNCaP+HOXB13 strain, the growth suppression rate was 42.1%±3.9% for siRNA against ACTB gene sequence, an essential human gene, and 18.1%±4.6% and 19.6%±0.3% (average ± standard deviation(SD)) for a siRNA against HOXB13 sequence No. 4 and No: 5 (siRNA No. 4 and siRNA No. 5, respectively). These results indicate that the introduction of HOXB13 gene, even partially, antagonizes specifically the growth suppression effect observed in prostate cancer cell PC-3 when siRNA against the HOXB13 gene is introduced. This suggests that HOXB13-siRNA exerts its growth suppression effect via suppression of HOXB13. From these results, it is believed that the HOXB13 gene relates to viability of not only prostate cancer cell LNCaP showing hormone dependent growth, but also hormone non-responsive PC-3 cell.

### Example 7 Colony Formation of HOXB13 Expressing NIH 3T3 Cell in Soft Agar

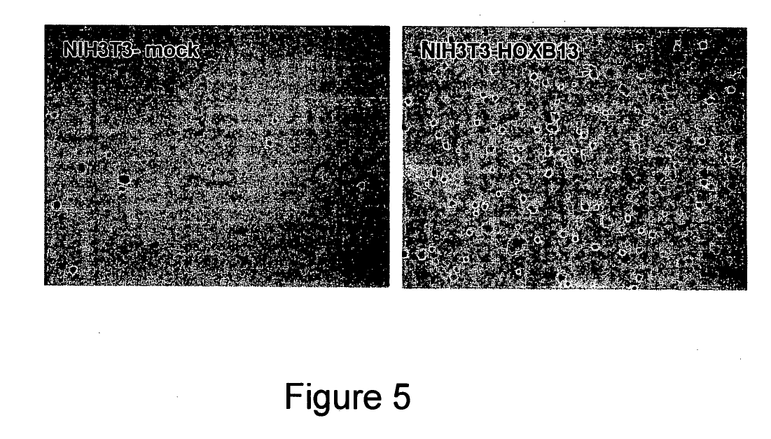

An established cell line stably expressing HOXB13 gene and a control cell strain, genetically unmodified parent cell, were collected by subjecting them to a trypsin-EDTA treatment, and washed with fresh medium twice. After that, 50,000 cell were warmed at about 38 to 39° C. per one well, suspended in 1 ml of RPMI 1640 medium containing 0.33% liquid Bactoagar and 20% of FCS, and immediately poured into a 12 well plate (3512: Corning Coaster) which RPMI 1640 medium containing 0.66% of Bactoagar (Difco) and 20% of FCS had been dispensed and solidified. The three same wells were prepared for each cell. Plates were left at room temperature for 30 minutes, allowing the Bactoagar to solidify completely, and incubated under the condition of 5% CO₂ and 37° C. for 21 days. To stain living cells, a MTT (3-(4,5-Dimethyl-2-thiazolyl)-2,5-dihphenyl-2H-tetrazolium bromide) 5 mg/ml solution was added to the plate, then the plate was incubated at 37° C. for 7 hours, washed with PBS buffer three times and microphotographs were taken (Nikon, DIAPHOTO300). The results are presented in FIG. 5. More MTT stained cells could be observed in the NIH3T3-HOXB13 strain cultured in the soft agar than in the NHI3T-mock, in which only the vector was introduced and this suggests that HOXB13 enhances viability of cells at anchorage independent growth.

What is claimed is:

1. A cancer therapeutic and/or preventive pharmaceutical composition comprising an oligonucleotide having at least one nucleotide sequence selected from the group consisting of
(a) a nucleotide sequence containing nucleotide numbers 177 to 1031 of SEQ ID NO: 1 of the Sequence Listing,
(b) a nucleotide sequence containing nucleotide numbers 131 to 985 of SEQ ID NO: 3 of the Sequence Listing,
(c) a nucleotide sequence containing nucleotide numbers 55 to 909 of SEQ ID NO: 5 of the Sequence Listing, and
(d) a nucleotide sequence containing nucleotide numbers 87 to 941 of SEQ ID NO: 7 of the Sequence Listing, or a nucleotide sequence complementary to a partial sequence thereof.

2. A cancer therapeutic and/or preventive pharmaceutical composition comprising an antibody which specifically recognizes HOXB13.

3. A cancer therapeutic and/or preventive pharmaceutical composition comprising a siRNA for at least one nucleotide sequence selected from the group consisting of
(a) the nucleotide sequence containing nucleotide numbers 177 to 1031 of SEQ ID NO: 1 of the Sequence Listing,
(b) the nucleotide sequence containing nucleotide numbers 131 to 985 of SEQ ID NO: 3 of the Sequence Listing,
(c) the nucleotide sequence containing nucleotide numbers 55 to 909 of SEQ ID NO: 5 of the Sequence Listing, and
(d) the nucleotide sequence containing nucleotide numbers 87 to 941 of SEQ ID NO: 7 of the Sequence Listing, or a partial sequence thereof.

4. A siRNA selected from the group consisting of
(a) a siRNA comprising the combination of an oligonucleotide of the nucleotide sequence of SEQ ID NO: 13 of the Sequence Listing, and an oligonucleotide of the nucleotide sequence of SEQ ID NO: 14 of the Sequence Listing;
(b) a siRNA comprising the combination of an oligonucleotide of the nucleotide sequence of SEQ ID NO: 15 of the Sequence Listing, and an oligonucleotide of the nucleotide sequence of SEQ ID NO: 16 of the Sequence Listing;
(c) a siRNA comprising the combination of an oligonucleotide of the nucleotide sequence of SEQ ID NO: 17 of the Sequence Listing, and an oligonucleotide of the nucleotide sequence of SEQ ID NO: 18 of the Sequence Listing;
(d) a siRNA comprising the combination of an oligonucleotide of the nucleotide sequence of SEQ ID NO: 27 of the Sequence Listing, and an oligonucleotide of the nucleotide sequence indicated in SEQ ID NO: 28 of the Sequence Listing; and
(e) a siRNA comprising the combination of an oligonucleotide of the nucleotide sequence of SEQ ID NO: 29 of the Sequence Listing, and an oligonucleotide of the nucleotide sequence of SEQ ID NO: 30 of the Sequence Listing.

5. A cancer therapeutic and/or preventive pharmaceutical composition comprising at least one siRNA selected from the group consisting of
(a) a siRNA comprising the combination of an oligonucleotide of the nucleotide sequence of SEQ ID NO: 13 of the Sequence Listing, and an oligonucleotide of the nucleotide sequence of SEQ ID NO: 14 of the Sequence Listing;
(b) a siRNA comprising the combination of an oligonucleotide of the nucleotide sequence of SEQ ID NO: 15 of the Sequence Listing, and an oligonucleotide of the nucleotide sequence of SEQ ID NO: 16 of the Sequence Listing;
(c) a siRNA comprising the combination of an oligonucleotide of the nucleotide sequence of SEQ ID NO: 17 of the Sequence Listing, and an oligonucleotide of the nucleotide sequence of SEQ ID NO: 18 of the Sequence Listing;
(d) a siRNA comprising the combination of an oligonucleotide of the nucleotide sequence of SEQ ID NO: 27 of the Sequence Listing, and an oligonucleotide of the nucleotide sequence of SEQ ID NO: 28 of the Sequence Listing; and
(e) a siRNA comprising the combination of an oligonucleotide of the nucleotide sequence of SEQ ID NO: 29 of the Sequence Listing, and an oligonucleotide of the nucleotide sequence of SEQ ID NO: 30 of the Sequence Listing, in combination with a pharmaceutically acceptable carrier.

6. A pharmaceutical composition according to any one of claims 1 to 3 and 5, wherein the cancer is prostate cancer.

7. A method of treating cancer comprising administering to a human in need thereof a pharmaceutically effective amount of the pharmaceutical composition according to any one of claims 1 to 3 and 5.

8. The method of treating cancer according to claim 7, wherein the cancer is prostate cancer.

9. A method of producing cancer cells comprising
(a) transforming cells using a polynucleotide selected from the group consisting of
(i) a polynucleotide of the nucleotide sequence containing nucleotides 177 to 1031 of SEQ ID NO: 1 of the Sequence Listing,
(ii) a polynucleotide of the nucleotide sequence containing nucleotides 131 to 985 of SEQ ID NO: 3 of the Sequence Listing,
(iii) a polynucleotide of the nucleotide sequence containing nucleotides 55 to 909 of SEQ ID NO: 5 of the Sequence Listing,
(iv) a polynucleotide of the nucleotide sequence containing nucleotides 87 to 941 of SEQ ID NO: 7 of the Sequence Listing, and
(v) a polynucleotide that hybridizes under stringent conditions with a polynucleotide of a nucleotide sequence complementary to said polynucleotide (i), (ii), (iii) or (iv), and contains a nucleotide sequence that encodes a protein substantially identical to HOXB13, and
(b) selecting a cell line in which a change in cell growth ability has occurred as a result of step (a).

10. The method of producing cancer cells according to claim 9, wherein the cells are animal cells.

11. The method of producing cancer cells according to claim 10, wherein the animal cells are derived from a mammal.

12. The method of producing cancer cells according to claim 11, wherein the mammal is a human, monkey, mouse or rat.

13. The method of producing cancer cells according to claim 11, wherein the mammal is a human.

14. The method of producing cancer cells according to claim 9, wherein the cells are mouse fibroblast cell line NIH3T3 cells.

15. The method of producing cancer cells according to any one of claims 9 to 14, wherein cells are transformed with a recombinant vector.

16. Cancer cells obtained by the method of producing cancer cells according to claim 9.

17. A non-human mammal introduced with the cancer cells according to claim 16.


SEQUENCE LISTING (32 sequences)

SEQ ID NO: 1 - DNA, 1316 bp, Homo sapiens
  CDS (177)..(1031)
tcctaatacgactcactatagggctcgagcggccgcccgggcaggtcgaatgcaggcgacttgcgagctgggagcgatttaaaacgctttggattcccccggcctgggtggggagagcgagctgggtgccccctagattccccgcccccgcacctcatgagccgaccctcggctccatggagcccggcaattatgccaccttggatggagccaaggatatcgaaggcttgctgggagcgggaggggggcggaatctggtcgcccactcccctctgaccagccacccagcggcgcctacgctgatgcctgctgtcaactatgcccccttggatctgccaggctcggcggagccgccaaagcaatgccacccatgccctggggtgccccaggggacgtccccagctcccgtgccttatggttactttggaggcgggtactactcctgccgagtgtcccggagctcgctgaaaccctgtgcccaggcagccaccctggccgcgtaccccgcggagactcccacggccggggaagagtaccccagtcgccccactgagtttgccttctatccgggatatccgggaacctaccacgctatggccagttacctggacgtgtctgtggtgcagactctgggtgctcctggagaaccgcgacatgactccctgttgcctgtggacagttaccagtcttgggctctcgctggtggctggaacagccagatgtgttgccagggagaacagaacccaccaggtcccttttggaaggcagcatttgcagactccagcgggcagcaccctcctgacgcctgcgcctttcgtcgcggccgcaagaaacgcattccgtacagcaaggggcagttgcgggagctggagcgggagtatgcggctaacaagttcatcaccaaggacaagaggcgcaagatctcggcagccaccagcctctcggagcgccagattaccatctggtttcagaaccgccgggtcaaagagaagaaggttctcgccaaggtgaagaacagcgctaccccttaagagatctccttgcctgggtgggaggagcgaaagtgggggtgtcctggggagaccagaaacctgccaagcccaggctggggccaaggactctgctgagaggcccctagagacaacacccttcccaggccactggctgctggactgttcctcaggagcggcctgggtacccagtatgtgcagggagacggaaccccatgtgacaggcccactccaccagggttcccaaagaacctggcccagtcataatcattcatcctcacagtggcaataatcacgataaccagt

SEQ ID NO: 2 - protein, 284 aa, Homo sapiens
MEPGNYATLDGAKDIEGLLGAGGGRNLVAHSPLTSHPAAPTLMPAVNYAPLDLPGSAEPPKQCHPCPGVPQGTSPAPVPYGYFGGGYYSCRVSRSSLKPCAQAATLAAYPAETPTAGEEYPSRPTEFAFYPGYPGTYHAMASYLDVSVVQTLGAPGEPRHDSLLPVDSYQSWALAGGWNSQMCCQGEQNPPGPFWKAAFADSSGQHPPDACAFRRGRKKRIPYSKGQLRELEREYAANKFITKDKRRKISAATSLSERQITIWFQNRRVKEKKVLAKVKNSATP

SEQ ID NO: 3 - DNA, 1270 bp, Homo sapiens
  CDS (131)..(985)
cgaatgcaggcgacttgcgagctgggagcgatttaaaacgctttggattcccccggcctgggtggggagagcgagctgggtgccccctagattccccgcccccgcacctcatgagccgaccctcggctccatggagcccggcaattatgccaccttggatggagccaaggatatcgaaggcttgctgggagcgggaggggggcggaatctggtcgcccactcccctctgaccagccacccagcggcgcctacgctgatgcctgctgtcaactatgcccccttggatctgccaggctcggcggagccgccaaagcaatgccacccatgccctggggtgccccaggggacgtccccagctcccgtgccttatggttactttggaggcgggtactactcctgccgagtgtcccggagctcgctgaaaccctgtgcccaggcagccaccctggccgcgtaccccgcggagactcccacggccggggaagagtaccccagtcgccccactgagtttgccttctatccgggatatccgggaacctaccacgctatggccagttacctggacgtgtctgtggtgcagactctgggtgctcctggagaaccgcgacatgactccctgttgcctgtggacagttaccagtcttgggctctcgctggtggctggaacagccagatgtgttgccagggagaacagaacccaccaggtcccttttggaaggcagcatttgcagactccagcgggcagcaccctcctgacgcctgcgcctttcgtcgcggccgcaagaaacgcattccgtacagcaaggggcagttgcgggagctggagcgggagtatgcggctaacaagttcatcaccaaggacaagaggcgcaagatctcggcagccaccagcctctcggagcgccagattaccatctggtttcagaaccgccgggtcaaagagaagaaggttctcgccaaggtgaagaacagcgctaccccttaagagatctccttgcctgggtgggaggagcgaaagtgggggtgtcctggggagaccagaaacctgccaagcccaggctggggccaaggactctgctgagaggcccctagagacaacacccttcccaggccactggctgctggactgttcctcaggagcggcctgggtacccagtatgtgcagggagacggaaccccatgtgacaggcccactccaccagggttcccaaagaacctggcccagtcataatcattcatcctcacagtggcaataatcacgataaccagt

SEQ ID NO: 4 - protein, 284 aa, Homo sapiens; identical to SEQ ID NO: 2

SEQ ID NO: 5 - DNA, 1026 bp, Homo sapiens
  CDS (55)..(909)
cgggtgccccctagattccccgcccccgcacctcatgagccgaccctcggctccatggagcccggcaattatgccaccttggatggagccaaggatatcgaaggcttgctgggagcgggaggggggcggaatctggtcgcccactcccctctgaccagccacccagcggcgcctacgctgatgcctgctgtcaactatgcccccttggatctgccaggctcggcggagccgccaaagcaatgccacccatgccctggggtgccccaggggacgtccccagctcccgtgccttatggttactttggaggcgggtactactcctgccgagtgtcccggagctcgctgaaaccctgtgcccaggcagccaccctggccgcgtaccccgcggagactcccacggccggggaagagtaccccagccgccccactgagtttgccttctatccgggatatccgggaacctaccacgctatggccagttacctggacgtgtctgtggtgcagactctgggtgctcctggagaaccgcgacatgactccctgttgcctgtggacagttaccagtcttgggctctcgctggtggctggaacagccagatgtgttgccagggagaacagaacccaccaggtcccttttggaaggcagcatttgcagactccagcgggcagcaccctcctgacgcctccgcctttcgtcgcggccgcaagaaacgcattccgtacagcaaggggcagttgcgggagctggagcgggagtatgcggctaacaagttcatcaccaaggacaagaggcgcaagatctcggcagccaccagcctctcggagcgccagattaccatctggtttcagaaccgccgggtcaaagagaagaaggttctcgccaaggtgaagaacagcgctaccccttaagagatctccttgcctgggtgggaggagcgaaagtgggggtgtcctggggagaccaggaacctgccaagcccaggctggggccaaggactctgctgagaggcccctagagacaacacc

SEQ ID NO: 6 - protein, 284 aa, Homo sapiens [sequence omitted]

SEQ ID NO: 7 - DNA, 1356 bp, Homo sapiens
  CDS (87)..(941)
ggattcccccggcctgggtggggagagcgagctgggtgccccctagattccccgcccccgcacctcatgagccgaccctcggctccatggagcccggcaattatgccaccttggatggagccaaggatatcgaaggcttgctgggagcgggaggggggcggaatctggtcgcccactcccctctgaccagccacccagcggcgcctacgctgatgcctgctgtcaactatgcccccttggatctgccaggctcggcggagccgccaaagcaatgccacccatgccctggggtgccccaggggacgtccccagctcccgtgccttatggttactttggaggcgggtactactcctgccgagtgtcccggagctcgctgaaaccctgtgcccaggcagccaccctggccgcgtaccccgcggagactcccacggccggggaagagtaccccagccgccccactgagtttgccttctatccgggatatccgggaacctaccagcctatggccagttacctggacgtgtctgtggtgcagactctgggtgctcctggagaaccgcgacatgactccctgttgcctgtggacagttaccagtcttgggctctcgctggtggctggaacagccagatgtgttgccagggagaacagaacccaccaggtcccttttggaaggcagcatttgcagactccagcgggcagcaccctcctgacgcctgcgcctttcgtcgcggccgcaagaaacgcattccgtacagcaaggggcagttgcgggagctggagcgggagtatgcggctaacaagttcatcaccaaggacaagaggcgcaagatctcggcagccaccagcctctcggagcgccagattaccatctggtttcagaaccgccgggtcaaagagaagaaggttctcgccaaggtgaagaacagcgctaccccttaagagatctccttgcctgggtgggaggagcgaaagtgggggtgtcctggggagaccaggaacctgccaagcccaggctggggccaaggactctgctgagaggcccctagagacaacacccttcccaggccactggctgctggactgttcctcaggagcggcctgggtacccagtatgtgcagggagacggaaccccatgtgacagcccactccaccagggttcccaaagaacctggcccagtcataatcattcatcctgacagtggcaataatcacgataaccagtactagctgccatgatcgttagcctcatattttctatctagagctctgtagagcactttagaaaccgctttcatgaattgagctaattatgaataaatttggaaaaaaaaaaaaaaaaaaaaaaaaaaaaaa

SEQ ID NO: 8 - protein, 284 aa, Homo sapiens
MEPGNYATLDGAKDIEGLLGAGGGRNLVAHSPLTSHPAAPTLMPAVNYAPLDLPGSAEPPKQCHPCPGVPQGTSPAPVPYGYFGGGYYSCRVSRSSLKPCAQAATLAAYPAETPTAGEEYPSRPTEFAFYPGYPGTYQPMASYLDVSVVQTLGAPGEPRHDSLLPVDSYQSWALAGGWNSQMCCQGEQNPPGPFWKAAFADSSGQHPPDACAFRRGRKKRIPYSKGQLRELEREYAANKFITKDKRRKISAATSLSERQITIWFQNRRVKEKKVLAKVKNSATP

SEQ ID NO: 9 - DNA, 26 bp, Artificial Sequence
  PCR sense primer for HOXB13
caccatggagcccggcaattatgcca

SEQ ID NO: 10 - DNA, 21 bp, Artificial Sequence
  PCR antisense primer for HOXB13
ttaaggggtagcgctgttctt

SEQ ID NO: 11 - DNA, 20 bp, Artificial Sequence
  RT-PCR sense primer for detecting HOXB13
ccaaaatgtcgtaacaactc

SEQ ID NO: 12 - DNA, 20 bp, Artificial Sequence
  RT-PCR antisense primer for detecting HOXB13
gaccttgatctgaacttctc

SEQ ID NO: 13 - DNA, 21 bp, Artificial Sequence
  siRNA No.1 sense strand for HOXB13
ggauaucgaaggcuugcugtt

SEQ ID NO: 14 - DNA, 21 bp, Artificial Sequence
  siRNA No.1 antisense strand for HOXB13
cagcaagccuucgauaucctt

SEQ ID NO: 15 - DNA, 21 bp, Artificial Sequence
  siRNA No.2 sense strand for HOXB13
guucaucaccaaggacaagtt

SEQ ID NO: 16 - DNA, 21 bp, Artificial Sequence
  siRNA No.2 antisense strand HOXB13
cuuguccuuggugaugaactt

SEQ ID NO: 17 - DNA, 21 bp, Artificial Sequence
  siRNA No.3 sense strand for HOXB13
ggugaagaacagcgcuacctt

SEQ ID NO: 18 - DNA, 21 bp, Artificial Sequence
  siRNA No.3 antisense strand for HOXB13
gguagcgcuguucuucacctt

SEQ ID NO: 19 - DNA, 21 bp, Artificial Sequence
  siRNA sense strand for Luciferase
cguacgcggaauacuucgatt

SEQ ID NO: 20 - DNA, 21 bp, Artificial Sequence
  siRNA antisense strand for Luciferase
ucgaaguauuccgcguacgtt

SEQ ID NO: 21 - DNA, 21 bp, Artificial Sequence
  siRNA sense strand for Eg5
cuggaucguaagaaggcagtt

SEQ ID NO: 22 - DNA, 21 bp, Artificial Sequence
  siRNA antisense strand for Eg5
cugccuucuuacgauccagtt

SEQ ID NO: 23 - DNA, 22 bp, Artificial Sequence
  RT-PCR sense primer for HOXB13
cttttggaaggcagcatttgca

SEQ ID NO: 24 - DNA, 25 bp, Artificial Sequence
  RT-PCR antisense primer for HOXB13
gtgatgaacttgttagccgcatact

SEQ ID NO: 25 - DNA, 19 bp, Artificial Sequence
  RT-PCR sense primer for GAPDH
gaaggtgaaggtcggagtc

SEQ ID NO: 26 - DNA, 20 bp, Artificial Sequence
  RT-PCR antisense primer for GAPDH
gaagatggtgatgggatttc

SEQ ID NO: 27 - DNA, 21 bp, Artificial Sequence
  siRNA No.4 sense strand for HOXB13
caguggcaauaaucacgautt

SEQ ID NO: 28 - DNA, 21 bp, Artificial Sequence
  siRNA No.4 antisense strand for HOXB13
aucgugauuauugccacugtt

SEQ ID NO: 29 - DNA, 21 bp, Artificial Sequence
  siRNA No.5 sense strand for HOXB13
guggcaauaaucacgauaatt

SEQ ID NO: 30 - DNA, 21 bp, Artificial Sequence
  siRNA No.5 antisense strand for HOXB13
uuaucgugauuauugccactt

SEQ ID NO: 31 - DNA, 21 bp, Artificial Sequence
  siRNA sense strand for ACTB
ugaagaucaagaucauugctt

SEQ ID NO: 32 - DNA, 21 bp, Artificial Sequence
  siRNA antisense strand for ACTB
gcaaugaucuugaucuucatt